(19) United States
(12) Patent Application Publication
(10) Pub. No.: US 2007/0128179 A1
(43) Pub. Date: Jun. 7, 2007

(54) Phospholipase(s) and use(s) thereof

(76) Inventors: Ponnampalam Gopalakrishnakone, Singapore (SG); Ramar Perumal Samy, Singapore (SG)

Correspondence Address: DICKSTEIN SHAPIRO LLP, 1825 EYE STREET NW, Washington, DC, 20006-5403, US

(21) Appl. No.: 11/594,173

(22) Filed: Nov. 8, 2006

Related U.S. Application Data
(60) Provisional application No. 60/734,294, filed on Nov. 8, 2005.

(51) Int. Cl. A61K 38/46; C12N 9/20

(52) U.S. Cl. 424/94.6; 435/198

(57) ABSTRACT

The present invention provides a method for the treatment and/or prevention of a bacterial related condition comprising administering to a subject a therapeutically effective amount of at least one phospholipase, isoform, derivative, mutant and/or fragment thereof. The phospholipase, isoform, derivative, mutant and/or fragment thereof, may be obtained from at least one venom selected from the group consisting of: Daboia russelli russelli, Daboia russelli siamensis, Daboia russelli pulchella, Crotalus adamanteus, Crotalus durissus terrificus, Pseudechis australis, Agkistrodon halys, Pseudechis guttata, Bitis arietans, Bitis gabonica rhinoceros, Echis carinatus, Acanthopis antarticus, Bungarus candidus, Bothrops asper, Bothrops jararacussu and/or Apis mellifera. The present invention also provides isolated peptides comprising at least one amino acid sequence selected from the group consisting of: SEQ ID NO:1, SEQ ID NO:2, an isoform, derivative, mutant and/or fragment thereof.

This application claims priority to U.S. Provisional Application Ser. No. 60/734,294, filed Nov. 8, 2005, the entire disclosure of which is incorporated by reference herein.

### FIELD OF THE INVENTION

The present invention relates to phospholipase(s) and use(s) thereof. The present invention also relates to phospholipase(s) used in the treatment and/or prevention of a bacterial related condition and/or disease.

### BACKGROUND OF THE INVENTION

Gram-negative Burkholderia pseudomallei, the causative agent of melioidosis, are found widely in soil and surface water throughout the tropics. High incidence of melioidosis has been found particularly in Southeast Asia and Northern Australia. A number of cases have been reported in Singapore, Malaysia, Thailand, Northern Australia, South China, Taiwan, South India, Africa and America. The majority of adult patients develop acute pulmonary or septicaemic illness with high mortality rates, or subacute melioidosis, characterized by multiple-abscess formation. In cases of septicaemic melioidosis, which is associated with a vigorous inflammatory cytokine response, septic shock continues to be a major cause of morbidity and mortality in patients. Tumor necrosis factor (TNF) is involved in the acquisition of melioidosis, and is also related to disease severity. Many agents have been used to treat septic shock, including monoclonal antibodies to endotoxins, II-1 receptor antagonists and various anti-inflammatory therapies but these have failed to produce effective results (Oscar Cirioni et al, 2002).

Antibiotic resistance has been of great concern during the last decades due to the extensive clinical use of classical antibiotics. Currently available antimicrobials fail to lower the mortality rate of melioidosis (Dance D A B, 1996; Leelarasamee A, 1998). B. pseudomallei demonstrate high levels of resistance to the action of cationic antimicrobial peptides such as polylysine, protamine sulfate, human neutrophil peptides (HNP-1), and polymyxins (Eickhoff T C et al, 1970; Jones A L et al, 1996). The above is just one example of bacteria which show resistance against currently available antimicrobials. Therefore, there is a need in the state of the art to develop antimicrobials with a new mechanism(s) of action which can potentially evade the emergence of drug resistance.

### SUMMARY OF THE IVNENTION

The present invention addresses the problems above. According to a first aspect, the present invention provides a method for the treatment and/or prevention of at least one bacterial related condition, wherein the method comprises administering to a subject a therapeutically effective amount of at least one phospholipase, isoform, derivative, mutant and/or fragment thereof. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. In particular, the related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli, with a proviso that the condition is not induced by Staphylococcus aureus and/or Escherichia coli when the phospholipase, isoform, derivative, mutant and/or fragment thereof is myotoxin II or BnpTx I. More in particular, the invention provides a method for the treatment and/or prevention of at least one bacterial related condition, wherein the condition is induced and/or caused by Burkholderia pseudomallei.

According to a particular aspect, the present invention provides a method for the treatment and/or prevention of at least one bacterial induced condition, wherein the method comprises administering to a subject a therapeutically effective amount of at least one phospholipase, isoform, derivative, mutant and/or fragment thereof comprising the amino acid sequence of SEQ ID NO:1 and/or SEQ ID NO:2. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli.

The condition may be melioidosis, septic shock and/or inflammation. The subject may be a mammal. In particular, the mammal may be human.

The phospholipase may be a secretory or cytoplasmic phospholipase. For example, the secretory phospholipase may be pancreatic, synovial and/or venomous phospholipase. The phospholipase, isoform, derivative, mutant and/or fragment thereof, may be from venom of one or more of the following, but not limited to Daboia russelli russelli, Daboia russelli siamensis, Daboia russelli pulchella, Crotalus adamanteus, Crotalus durissus terrificus, Pseudechis australis, Agkistrodon halys, Pseudechis guttata, Bitis arietans, Bitis gabonica rhinoceros, Echis carinatus, Acanthopis antarticus, Bungarus candidus, Bothrops asper, Bothrops jararacussu and/or Apis mellifera.

According to a further aspect, the phospholipase, isoform, derivative, mutant and/or fragment thereof may be phospholipase A₂. In particular, the phospholipase, isoform, derivative, mutant and/or fragment thereof may comprise at least one amino acid selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13 and SEQ ID NO:14. In particular, the phospholipase, isoform, derivative, mutant and/or fragment thereof has an amino acid sequence consisting of the sequence of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13 and/or SEQ ID NO:14.

The phospholipase, isoform, derivative, mutant and/or fragment thereof may comprise an isoform, derivative, mutant and/or fragment thereof of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13 and/or SEQ ID NO:14. In particular, the phospholipase, isoform, derivative, mutant and/or fragment thereof may comprise an isoform, derivative, mutant and/or fragment thereof of a polypeptide consisting of the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13 and/or SEQ ID NO:14. The phospholipase, isoform, derivative, mutant and/or fragment thereof may comprise at least one amino acid substitution, addition, deletion and/or at least one chemical modification.

According to a further aspect, the phospholipase, isoform, derivative, mutant and/or fragment thereof may be administered in conjunction with at least one pharmaceutically acceptable excipient, diluent, carrier and/or adjuvant.

According to a second aspect, the present invention provides a pharmaceutical composition formulated for the treatment and/or prevention of at least one bacterial related condition, wherein the composition comprises: a therapeutically effective amount of: a phospholipase, isoform, derivative, mutant and/or fragment thereof; and/or at least one pharmaceutically acceptable excipient, diluent, carrier and/or adjuvant. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. In particular, the related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli, with the proviso that the condition is not induced by Staphylococcus aureus and/or Escherichia coli when the phospholipase, isoform, derivative, mutant and/or fragment thereof is myotoxin II or BnpTx I.

The phospholipase, isoform, derivative, mutant and/or fragment thereof, may be any suitable phospholipase, isoform, derivative, mutant and/or fragment thereof as described above. In particular, the phospholipase may be phospholipase A₂.

According to another aspect, the present invention also provides a kit for the treatment and/or prevention of at least one bacterial related condition, comprising a phospholipase, isoform, derivative, mutant and/or fragment thereof, and optionally at least one pharmaceutically acceptable excipient, diluent, carrier and/or adjuvant. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. In particular, the at least one condition is induced by at least one of the following: Burkholderia pseudomallei, Proteus vulgaris, Enterobacter aerogenes, Proteus mirabilis, Pseudomonas aeruginosa, Staphylococcus aureus and Escherichia coli, with the proviso that the condition is not induced by Staphylococcus aureus and/or Escherichia coli when the phospholipase, isoform, derivative, mutant and/or fragment thereof is myotoxin II or BnpTx I.

The phospholipase, isoform, derivative, mutant and/or fragment thereof, may be any suitable phospholipase, isoform, derivative, mutant and/or fragment thereof, as described above.

The present invention also provides an isolated peptide comprising at least one amino acid sequence selected from the group consisting of: SEQ ID NO:1, SEQ ID NO:2, an isoform, derivative, mutant and/or fragment thereof. The peptide may be isolated and/or purified from biological material, expressed from recombinant DNA, and/or prepared by chemical synthesis.

The peptide according to the present invention may be isolated and/or purified from venom. In particular, the peptide is isolated and/or purified from the venom of Daboia russelli russelli. Even more in particular, the molecular weight of the peptide is 13822 Da or 13669 Da.

The peptide may be a fused peptide and may comprise at least one peptide comprising the sequence of SEQ ID NO:1 and/or SEQ ID NO:2.

According to another aspect, there is provided at least one isolated and/or purified venom comprising peptides for treatment and/or prevention of a bacterial related condition. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. In particular, the condition is induced by Burkholderia pseudomallei (TES) and/or Burkholderia pseudomallei (KHW).

### BRIEF DESCRIPTION OF THE DRAWINGS

FIG. 1 shows the purification of Russell's viper (Daboia russelli russelli) venom by gel filteration chromatography and the SDS-PAGE profile of the purified fractions. FIG. 1(A) represents fractionation of Russell's viper (Daboia russelli russelli) venom (1 gm/10 ml) on a Superdex-G75 column (280 nm) with 50 mM Tris-HCl buffer (pH 7.4). FIG. 1(B-C) shows further purification done on a reverse-phase Jupitor C18 column (AKTA explorer, Amarsham Pharmacia Biotech, Sweden), eluted with a linear gradient of 80% acetonitrile in 0.1% trifluroacetic acid. Elution of protein was monitored at and 215 nm. FIG. 1(D) shows the final fraction obtained by RP-HPLC using C18 column. FIG. 1(E) represents the determination of molecular mass for the final fraction by MALDI-TOF/MS. FIG. 1(F-I) shows the Sodium dodecyl sulphate-Polyacrylamide gel electrophoresis SDS-PAGE profile of whole venoms, RP-HPLC fractions and purity checked for the final fraction of Daboia toxin and mass determined as 15 kDa.

FIG. 2 represents the antimicrobial activities of crude venoms (each disc contained 20 μl of 100 μg/ml) of different snake species tested against gram-negative Burkholderia pseudomallei. Following 24 h incubation at 37° C., zone of inhibition given by each venom was compared with that of the standard drug chloramphenicol (30 μg/disc). FIG. 2(A) shows the inhibition zones against Burkholderia pseudomallei given by Daboia russelli russelli, venom. FIG. 2(B) shows the inhibition zones against Burkholderia pseudomallei given by Agkistrodon halys venom. FIG. 2(C) shows the inhibition zones against Burkholderia pseudomallei given by Crotalus adamanteus venom. FIG. 2(D) shows the inhibition zones against Burkholderia pseudomallei given by Bitis gabonicarhinoceros venom. FIG. 2(E) shows rough wrinkled morphological features of gram-negative Burkholderia pseudomallei bacteria after 36 h incubation of bacilli grown on Tryptic Soy agar plates at 37° C. FIG. 2(F) shows rough wrinkled morphological features of gram-negative Burkholderia pseudomallei bacteria after 72 h incubation of bacilli grown on Tryptic Soy agar plates at 37° C.

FIG. 3 shows the antimicrobial activity of purified fractions of venoms on a range of bacteria. FIG. 3(A) shows the activity of crude venoms of D. russelli russelli against Staphylococcus aureus. FIG. 3(B) shows the activity of DRR2-PLA₂ against Staphylococcus aureus. FIG. 3(C) shows the activity of DRR2-PLA₂ active Enterobacter aerogenes. FIG. 3(D) represents the antimicrobial activities of fractions (A13, A14, A15), purified from the crude venom by size exclusion chromatographic separation (superdex G75 160×4 cm) on Staphylococcus aureus. Zones of inhibition by Daboia russelli russelli. FIG. 3(E) represents the antimicrobial activities of fractions (B13, B14, B15), purified Gel filtration. FIG. 3(F) represents the antimicrobial activities of gel filtration fractions (C1, C2, C3) against Staphylococcus aureus. FIG. 3(G) and FIG. 3(H) depict the in vitro antimicrobial activity of purified enzyme DRR2-PLA₂ of Daboia russelli russelli against bacteria (S. aureus, P. vulgaris, P. mirabilis, E. coli, E. aerogenes). FIGS. 3(I) and 3(J) depict the in vitro antimicrobial activity of purified enzyme DRR2-PLA₂ of Daboia russelli russelli against multi-drug resistant B. pseudomallei strains.

FIG. 4 represents phospholipase A₂ activity against pathogen from patients with KHW. Micro-dilution technique was used to test the MICs of PLA₂s as compared to that of the antibiotics. The values are the optical density read at 560 nm (means±S.D.) from a single experiment performed in triplicates.

FIG. 5 shows the phospholipase A2 activity against pathogen from patients with TES. Micro-dilution technique was used to test the MICs of PLA₂s as compared to that of the antibiotics. The values are the optical density read at 560 nm (means±S.D.) from a single experiment performed in triplicates.

FIG. 6 shows the minimum inhibitory concentrations (MICs) of DRR2-PLA₂ phospholipase A₂ enzymes was determine d by broth-dilution assay against Gram-negative and Gram-positive bacteria by increasing concentrations. FIG. 6(A) shows DRR2-PLA₂ kills Staphylococcus aureus in a dose-dependent fashion. The activity of PLA₂ was 10-fold greater against S. aureus. FIG. 6(B & C) shows PLA₂ inhibit Proteus vulgaris (MICs) and Proteus mirabilis (MICs) effectively than the (D & E) Enterobacter earogenes and Escherichia coli. FIG. 6(F) shows the inhibitory effect was very weak against Pseudomonas aeruginosa.

FIG. 7 shows that cell proliferation (U-937 Human macrophage cell line) was determined by XTT assay to evaluate the cytotoxic effect of different venoms FIG. 7(a) South American rattlesnake (Crotalus adamanteus), FIG. 7(b) Russells viper (Daboia russelli russelli), FIG. 7(c) King brown (Pseudechis australis), FIG. 7(d) Pallas (Agkistrodon halys), FIG. 7(e) Speckled brown (Pseudechis guttata), FIG. 7(f) Crotoxin B (Crotalus durissus terrificus), FIG. 7(g) Daboiatoxin (Daboia russelli russelli), FIG. 7(h) Melittin (Apis mellifera), FIG. 7(i) Mulgatoxin (Pseudechis australis), FIG. 7(j) Crotoxin A (Crotalus durissus terrificus). The macrophages were incubated with varying concentrations of venoms (0.05-10 mg/ml) and PLA₂s (0.05-10 μg/ml).

FIG. 8: Morphological changes of U-937 Human macrophage cell line after exposure to Crotalus adamanteus venom at different concentrations. (Ctrl) macrophage supplemented with medium without any treatment served as control; (PC), cells exposed with ceftazidime as a positive control. Macrophages were incubated with venom at different (0.05-10 mg/mL).

FIG. 9: Morphological changes of U-937 Human macrophage cell line after exposure to PLA₂s at varying concentrations (0.05-10 μg/mL). (Ctrl), control macrophages supplemented with medium without any treatment; (PC), cells exposed with ceftazidime as a positive control.

FIG. 10: (A & B) shows the cell proliferation of THP-1 cells at various concentrations of DRR2-PLA₂. Ctrl-1 and Ctrl-2 are negative controls and PC-1 and PC-2 are positive control, NC contained cell culture media only without any cells as a normal control. Ctrl-1 contained cells only while Ctrl-2 contained cells and B. pseudomallei. PC-1 THP-1 cells treated with 10% Triton ×100 used as a positive control, PC-2 THP-1 cells treated with Ceftazidime as a drug control.

FIGS. 11(a) to (h): These figures show the morphological changes of THP-1 cells (Human macrophage) at varying concentrations of DRR2-PLA₂.

FIG. 12: (A) to (F) shows the LDH assay of THP-1 cells at various concentrations of DRR2-PLA₂ in the presence of different bacteria strains.

FIG. 13: Hemolytic activity of (A) DRR1-PLA₂, (B) DRR2-PLA₂ were incubated with Human erythrocytes with the different concentrations (10-125 μM) of enzymes and hemolytic was measured. Values are percentages of hemolytic in the absence of antimicrobial peptides and 100% significant hemolytic activity was seen at 10% Triton X-100 used as a positive control (PC).

FIG. 14: Comparison of the amino acid sequences of Crotoxin basic chain 1, CB1 [Crotalus durissus terrificus] phospholipase A₂ enzymes (Phosphatidylcholine 2-acylhydrolase) with other PLA₂ₛ of Mojave toxin basic chain, Mtx-b [Crotalus scutulatus scutulatus], Crotoxin basic chain 2, CB2 [Crotalus durissus terrificus], Agkistrotoxin, ATX [Agkistrodon halys], VRV-PL-Villa [Daboia russelli pulchella], BOTAS (Myotoxin I) Bothrops asper (Terciopelo), ATXA, ammodytoxin A precursor [Vipera ammodytes ammodytes]; RVV-VD [Daboia russelli russelli]; RV-4 precursor [Daboia russelli siamensis], BOTAS (Myotoxin II) [Bothrops asper], BOTJR (BthTX-I) [Bothrops jararacussu], ECHCA (Ecarpholin S) [Echis carinatus (Saw-scaled viper)], Crotoxin acid chain precursor (CA) [Crotalus durissus terrificus], β-bungarotoxin A6 chain precursor [Bungarus multicinctus (Many-banded krait)] and OXYSC taipoxin alpha chain [Oxyuranus scutellatus scutellatus]. Completely conserved residues in all sequences are bolded and marked by asterisks. The gaps are inserted in the sequences in order to attain maximum homology.

FIG. 15: Hydropathic profiles of phospholipase A₂ enzyme such as crotoxin b, daboiatoxin, mulgatoxin, taipoxin, ammodytoxin A, bee venom PLA₂, beta-bungarotoxin and mojavetoxin were calculated by using the kyte-doolittle method.

FIG. 16: (A & B) Comparison of MICs of DRR1-PLA₂ and DRR2-PLA₂ enzymes against B. pseudomallei (strain KHW). FIG. 16(C-F). Scanning electron microscopic pictures of Burkholderia pseudomallei after the treatment with DRR2-PLA₂ compared to control.

FIG. 17: The microarray analysis data showing differential expression of genes following treatment of THP-1 cells with DRR2-PLA₂.

FIG. 18: (A to L) Graphical representation of differential expression of genes following treatment of THP-1 cells.

FIGS. 19 to 22: SEM analysis of various bacteria in the presence or absence of DRR2-PLA₂.

FIG. 23: (A & B) Molecular mass determination of PLA₂ isolated from A. halys venom using MALDI-TOF.

### BRIEF DESCRIPTION OF THE SEQUENCES

SEQ ID NO:1:
SLLGFGCMILEETGVMIELEKNCNQHPE;
SEQ ID NO:2:
SLLEFGMMILEETGKLAVPFYSKYGLYCGCGGKTPDD;
SEQ ID NO:3:
HLLQFNKHIKFETRKNAIPFYAFYGCYCGWGGRGRPKDATRDCCFVHDCC
YGKLAKCNTKWDIYPYSLKSGYITCGKGTWCEEQICECDRVAAECLRRSL
STYKYGYHFYPDSRCRGPSETC;
SEQ ID NO:4:
SLLQFNKMIKFETRKNAUPFYAFYGCYCGWGGQGRPKDATDRCCFUHDCC
YGKLAKCNTKWDIYRYSLKSGYITCGKGTWCKEQICECDRVAAECLRRSL
STYKNEYMFYPDSRCREPSETC;
SEQ ID NO:5:
NLLQFNKMIKEETGKNAIPFYAFYGCYCGGGGQGKPKDGTDRCCFUHDCC
YGRLVNCNTKSDIYSYSLKEGYITCGKGTNCEEQICECDRVAAECFRRNL
DTYNNGYMFIRDSKCTETSEEC;
SEQ ID NO:6:
LLEFGKMILEETGKLAIPSYSSYGCYCGWGGKGTPKDATDRCCFVHDCCY
GNLPDCNPKSDRYKYKRVNGAIVCEKGTSCENRICECDKAAAICFRQNLN
TYSKKYMLYPDFLCKG;
SEQ ID NO:7:
LIEFAKMILEETKRLPFPYYTTYGCYCGWGGQGQPKDATDRCCFVHDCCY
GKLSNCKPKTDRYSYSRKSGVIICGEGTPCEKQICECDKAAAVCFRENLR
TYKKRYMAYPDLLCKKPAEKC;
SEQ ID NO:8:
NLFQFAEMIVKMTGKNPLSSYSDYGCYCGWGGKGKPQDAIDRCCFVHDCC
YEKVKSCKPKLSLYSYSFQNGGIVCGDNHSCKRAVCECDRVAATCFRDNL
NTYDKKYHNYPPSQCTGTEQC;
SEQ ID NO:9:
NLFQFARMINGKLGAFSVWNYISYGCYCGWGGQGTPKDATDRCCFVHTCC
YGGVKGCNPKLAYICYSFQRGNIVCGRNNGCLRTICECDRVAANCFHQNK
NTYNKEYKFLSSSKCRQRSEQC;
SEQ ID NO:10:
LFELGKMILQETGKNPAKSYGAYGCNCGVLGRGKPKDATDRCCYVHKCCY
KKLTGCNPKKDRYSYSWKDKTIVCGENNSCLKELCECDKAVAICLRENLN
TYNKKYRYYLKPLCK;
SEQ ID NO:11:
LFELGKMILQETGKNPAKSHGAYGCNCGVLGRGKPKDATDRCCYVHKCCY
KKLTGCDPKKDRYSYSWKDKTIVCGENNPCLKELCECDKAVAICLRENLG
IYNKKYRYHLKPFCK;
SEQ ID NO:12:
WELGKMIIQETGKSPFPSYTSYGCFCGGGERGPPLDATDRCCLAHSCCYD
TLPDCSPKTDRYKYKRENGEIICENSTSCKKRICECDKAVAVCLRKNLNT
YNKKYTYYPNFWCKGDIEKC;
SEQ ID NO:13:
HLLQFRKMIKKMTGKEPVVSYAFYGCY;
SEQ ID NO:14:
IVSPPVCGNELLEVGEECDD.

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

The present invention provides phospholipase and uses thereof. Phospholipases are peptides which are highly structured mini-proteins that are small size. Their structural stability and target specificity make them important pharmacological probes. For example, secretory phospholipase A₂ (PLA₂) enzymes that catalyse the hydrolysis of the sn-2 acyl bond of glycerophospholipids to produce free fatty acids and lysophospholipids are implicated in a range of diseases. Further, because of the fact that cytokines are involved in pathogenesis and progression of diseases such as melioidosis, and that phospholipase plays an important role in the regulation of cytokines, phospholipases such as PLA₂ may be useful as an alternative treatment for septicemic melioidosis.

Melioidosis is a life threatening diseases which is endemic in Southeast Asia and Northern Australia. Melioidosis and cases of infection have been reported in military personnel of various countries in the region. Four deaths out of 23 diagnosed cases of Burkholderia pseudomallei (B. pseudomallei) infection in military personnel were reported in Singapore (Heng B H et al, 1998). The prevalence of the infection in Singapore was 0.2% in the military as well as civilian population. Mortality rate of septicaemic melioidosis used to occur 87% worldwide. In cases of septicaemic melioidosis, septic shock continues to be a major cause of morbidity and mortality in patients. Currently available antimicrobials failed to lower the mortality rate of melioidosis (Leelarasamee A, 1998).

Therefore, the search for effective bactericidal peptides from venoms has been an important are of active research against bacteria such as B. pseudomallei. The antimicrobial peptides are ubiquitous in nature as part of the innate immune system and hot defense mechanisms.

Accordingly, a first aspect of the present invention is a method for the treatment and/or prevention of at least one bacterial related condition, comprising administering to a subject a therapeutically effective amount of at least one phospholipase, isoform, derivative, mutant and/or fragment thereof. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. In particular, the related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli, with the proviso that the condition is not induced by Staphylococcus aureus and/or Escherichia coli when the phospholipase, isoform, derivative, mutant and/or fragment thereof is myotoxin II or BnpTx I. In particular, the condition is induced by Burkholderia pseudomallei (KHW) or Burkholderia pseudomallei (TES).

The present invention also provides a method for the treatment and/or prevention of at least one bacterial related condition comprising administering to a subject a therapeutically effective amount of at least one phospholipase, isoform, derivative, mutant and/or fragment thereof comprising the amino acid sequence of SEQ ID NO:1 and/or SEQ ID NO:2. For example, the condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. In particular, the condition is induced by Burkholderia pseudomallei (KHW) or Burkholderia pseudomallei (TES).

The condition according to any aspect of the present invention may be melioidosis, septic shock and/or inflammation. The subject may be a mammal. In particular, the mammal may be a human.

According to any aspect of the present invention, the phospholipase, isoform, derivative, mutant and/or fragment thereof, may be a secretory phospholipase or cytoplasmic phospholipase. The secretory phospholipase may be pancreatic, synovial and/or venomous phospholipase.

In particular, the phospholipase may be from the venom of Daboia russelli russelli, Daboia russelli siamensis, Daboia russelli pulchella, Crotalus adamanteus, Crotalus durissus terrificus, Pseudechis australis, Agkistrodon halys, Pseudechis guttata, Bitis arietans, Bitis gabonica rhinoceros, Echis carinatus, Acanthopis antarticus, Bungarus candidus, Bothrops asper, Bothrops jararacussu and/or Apis mellifera.

Accordingly, the phospholipase, isoform, derivative, mutant and/or fragment thereof according to the invention may be from snake, scorpion or spider venom. In particular, the phospholipase, isoform, derivative, mutant and/or fragment thereof is from Daboia russelli russelli, Crotalus durissus terrificus, Agkistrodon halys, Bothrops asper, Daboia russelli siamensis, Bothrops jararacussu, Echis carinatus venom.

According to any aspect of the present invention, phospholipase may also include phospholipase from all types, isoforms, derivatives, groups, and subgroups of phospholipases, including but not limited to phospholipase A₁, phospholipase A₂, phospholipase B, phospholipase C or phospholipase D.

The phospholipase, isoform, derivative, mutant, and/or fragment may comprise at least one amino acid substitution, addition, deletion, and/or at least one chemical modification. An isoform, derivative, mutant and/or fragment of phospholipase may be defined as at least one polypeptide with an amino acid sequence substantially identical with phospholipase, with conserved amino acid changes that may be made without altering the function of the phospholipase according to the invention.

Particularly, it is within the knowledge of a skilled person how to make amino acid substitutions, for example at least one conservative amino acid substitution without altering the phospholipase's biological, pharmaceutical and/or therapeutic activity. In particular, a “conservative amino acid substitution” is one in which the amino acid residue is replaced with an amino acid residue having a similar side chain. Families of amino acid residues having similar side chains have been defined in the art. These families include hydrophobic, hydrophilic, basic and acid amino acids as described below. Thus, a predicted non-essential amino acid residue in the peptide of the invention is preferably replaced with another amino acid residue from the same category. For example, a hydrophobic amino acid is replaced by another hydrophobic amino acid, etc.

A “non-essential” amino acid residue is a residue that can be altered from the wild-type sequence of a phospholipase without abolishing or substantially altering the phospholipase activity. Preferably the alteration does not substantially alter the phospholipase activity, e.g., the activity is at least 20%, 40%, 60%, 70% or 80% of wild-type. An “essential” amino acid residue is a residue that, when altered from the wild-type sequence of a phospholipase, results in abolishing phospholipase activity such that less than 20% of the wild-type activity is present. For example, conserved amino acid residues in between the phospholipase, e.g., the phospholipases as described in Jeyaseelan et al., 2000 are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Hydrophobic” amino acids are: A, V, L, I, P, W, F and M; “hydrophilic” amino acids are: G, S, T, Y, C, N and Q; however Y has a hydrophobic aromatic group and may be considered to be acting as a hydrophobic amino acid depending on the circumstances and uses; “basic” amino acids are: K, R and H; and “acidic” amino acids are: D and E.

Alternatively, in another embodiment, mutations can be introduced randomly along all or part of the coding sequence of the nucleic acid encoding the peptide of the invention, such as by saturation mutagenesis, and the resultant mutants can be screened for anticoagulant activity to identify mutants that retain that activity.

Thus, a predicted non-essential amino acid residue in a phospholipase protein is preferably replaced with another amino acid residue from the same side chain family. Alternatively, in another embodiment, mutations can be introduced randomly along all or part of a phospholipase coding sequence, such as by saturation mutagenesis, and the resultant mutants can be screened for phospholipase biological activity to identify mutants that retain activity.

Amino acid residues can be generally sub-classified into major subclasses as follows:
Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.
Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.
Charged: The residues are charged at physiological pH and, therefore, include amino acids having acidic or basic side chains (i.e., glutamic acid, aspartic acid, arginine, lysine and histidine).
Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.
Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterises certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon.

Several amino acid similarity matrices (e.g., PAM120 matrix and PAM250 matrix as disclosed for example by Dayhoff et al. (1978), A model of evolutionary change in proteins. Matrices for determining distance relationships in M. O. Dayhoff, Atlas of protein sequence and structure, Vol. 5, pp. 345-358, National Biomedical Research Foundation, Washington, D.C.; and in Gonnet et al., 1992, Science 256(5062): 144301445), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behaviour.

Amino acid residues can be further sub-classified as cyclic or noncyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always nonaromatic.

The gene-encoded secondary amino acid proline is a special case due to its known effects on the secondary conformation of peptide chains, and is not, therefore, included in a group.

The “modified” amino acids that may be included in the phospholipases are gene-encoded amino acids which have been processed after translation of the gene, e.g., by the addition of methyl groups or derivatisation through covalent linkage to other substituents or oxidation or reduction or other covalent modification. The classification into which the resulting modified amino acid falls will be determined by the characteristics of the modified form. For example, if lysine were modified by acylating the .epsilon.-amino group, the modified form would not be classed as basic but as polar/large.

Certain commonly encountered amino acids, which are not encoded by the genetic code, include, for example, β-alanine (β-Ala), or other omega-amino acids, such as 3-aminopropionic, 2,3-diaminopropionic (2,3-diaP), 4-aminobutyric and so forth, α-aminoisobutyric acid (Aib), sarcosine (Sar), omithine (Om), citrulline (Cit), t-butylalanine (t-BuA), t-butylglycine (t-BuG), N-methylisoleucine (N-Melle), phenylglycine (Phg), and cyclohexylalanine (Cha), norleucine (Nle), 2-naphthylalanine (2-Nal); 1,2,3,4-tetrahydroisoquinoline-3-carboxylic acid (Tic); β-2-thienylalanine (Thi); methionine sulfoxide (MSO); and homoarginirie (Har). These also fall conveniently into particular categories.

Based on the above definitions, Sar, beta-Ala and Aib are small; t-BuA, t-BuG, N-Melle, Nle, Mvl, Cha, Phg, Nal, Thi and Tic are hydrophobic; 2,3-diaP, Om and Har are basic; Cit, Acetyl Lys and MSO are neutral/polar/large. The various omega-amino acids are classified according to size as small (β-Ala and 3-aminopropionic) or as large and hydrophobic (all others).

Other amino acid substitutions for those encoded in the gene can also be included in SLEs within the scope of the invention and can be classified within this general scheme according to their structure.

In a further aspect, the phospholipase may comprise a “biologically active portion” of a phospholipase, which is defined as including a fragment of a phospholipase protein, which participates in an interaction, e.g., an intra-molecular or an inter-molecular interaction. An inter-molecular interaction can be a specific binding interaction or an enzymatic interaction (e.g., the interaction can be transient and a covalent bond is formed or broken). An inter-molecular interaction can be between a phospholipase molecule and a non-phospholipase molecule, or between a first phospholipase molecule, (e.g., a light chain of a phospholipase) and a second phospholipase molecule (e.g., a dimerization interaction). Biologically active portions of a phospholipase protein include peptides comprising amino acid sequences sufficiently homologous to or derived from the amino acid sequence of the phospholipase protein, and exhibits at least one activity of a phospholipase protein. Typically, biologically active portions comprise a domain or motif with at least one activity of the phospholipase protein. A biologically active portion of a phospholipase protein can be a polypeptide which is, for example, 10, 25, 50, 100, 200 or more amino acids in length. Preferably, said fragment is a “biologically-active portion” having no less than 1%, preferably no less than 10%, more preferably no less than 25% and even more preferably no less than 50% of the processing activity of at least one phospholipase described herein.

There is provided a “fragment” of a phospholipase of the invention. The term “fragment” includes within its scope heavy and light chain fragments of a phospholipase.

In particular, the phospholipase, isoform, derivative, mutant and/or fragment thereof, may be phospholipase A₂. The phospholipase A₂ may be selected from any one of Group I to Group XI phospholipase A₂.

The phospholipase, isoform, derivative, mutant and/or fragment thereof, may comprise at least one amino acid selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13 and SEQ ID NO:14 SEQ ID NO:12. Alternatively, the phospholipase, isoform, derivative, mutant and/or fragment thereof, may comprise an amino acid having the amino acid sequence of at least one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13 and SEQ ID NO:14.

According to any aspect of the present invention, the phospholipase, isoform, derivative, mutant and/or fragment thereof comprises an isoform, derivative, mutant and/or fragment thereof of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13 and/or SEQ ID NO:14.

According to a particular aspect, the condition is not induced by Staphylococcus aureus and/or Escherichia coli when the phospholipase, isoform, derivative, mutant and/or fragment thereof is myotoxin II or BnpTx I.

The phospholipase A₂ (PLA₂) superfamily is defined as a broad range of enzymes with the ability to catalyze the hydrolysis of the middle (sn-2) ester bond of substrate phospholipids.

An enzyme is assigned to a phospholipase A₂ group according to four criteria, the first essential criterion being that it must catalyze the hydrolysis of the sn-2 ester bond of a phospholipid substrate. Naturally occurring substrates include platelet activating factor, short fatty acid chain oxidized phospholipids, and long fatty acid chain phospholipids, with sn-2 acyl chains ranging from two (acetyl) to 20 carbons (arachidonate) and even longer. While the major activity must be PLA₂ activity, members of the PLA₂ superfamily may possess other activities, such as PLA₁, lysophospholipase A₁/ A₂, acyl transferase, or transacylase activity.

The second essential criterion for an enzyme to be assigned to a PLA₂ Group is that the complete amino acid sequence for the mature protein is known. Future additions to the PLA₂ superfamily should be cloned, expressed, and purified to correlate the sequence to specific activity in an unambiguous system, regardless of whether they are discovered by DNA- or activity-based searches.

The third criterion for the classification is that each PLA₂ group should include all of those enzymes which have readily identifiable sequence homology. Specifically, if more than one homologous PLA₂ gene exists within a species (paralogs), then each PLA₂ gene is assigned a subgroup letter, as in the case of Groups IVA, IVB, and IVC PLA₂. It is also possible that paralogs will exist only in certain species, as is the case with Group IIC PLA₂. Homologs from different species (orthologs) are classified within the same subgroup wherever such assignments are possible, such as for zebra fish and human Group IVA PLA₂.

The fourth criterion for classification considers active splice variants of the same PLA₂ gene to be distinct proteins, but part of the same subgroup. Each splice variant with confirmed activity is numbered, for example, for Group VIA PLA₂, which has two confirmed, active splice variants, referred to as Group VIA-1 PLA₂ and Group VIA-2 PLA₂. For inactive splice variants, for example in the case of Group VIA PLA₂, the variants are referred to not as PLA₂ enzymes, but still using the Group nomenclature, as Group VIA Ankyrin-1 and Group VIA Ankyrin-2.

Phospholipase A₂ may be defined as an enzyme, comprising a polypeptide which is characterised by an amino acid sequence which is coded for by a phospholipase A₂ gene. Particularly, phospholipase A₂ may be defined as an enzyme comprising at least one amino acid sequence selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13 and SEQ ID NO:14, which is coded for by a phospholipase A₂ gene.

In a further aspect, phospholipase A₂ (PLA₂) may be from other organisms, in particular pancreatic and secretory PLA₂ from mammals (e.g. human, bovine, rat, canine), or from bee and other venoms. Secreted PLA₂ (sPLA₂) is a phospholipase A₂ enzyme that is expressed in an extracellular secretion, for example, mammalian pancreatic sPLA₂, (e.g. human, bovine, rat, canine), mammalian synovial sPLA₂, and venom sPLA₂ (e.g. from bee, snakes, crotalids and elapids). In particular, secreted PLA₂ may be from venom from Daboia russelli russelli (SEQ ID NO:1, SEQ ID NO:2, SEQ ID NO:8), Crotalus durissus terrificus (SEQ ID NO:3, SEQ ID NO:4), Agkistrodon halys (SEQ ID NO:5, SEQ ID NO:13, SEQ ID NO:14), Daboia russelli pulchella (SEQ ID NO:6), Bothrops asper (SEQ ID NO:7, SEQ ID NO:10), Daboia russelli siamensis (SEQ ID NO:9), Bothrops jararacussu (SEQ ID NO:11) or Echis carinatus (SEQ ID NO:12).

Synovial sPLA₂ is a secreted phospholipase A₂ which is isolated from synovial fluid, which is defined as a clear, viscid substance formed as a diasylate, containing protein. Cells of the intima and the vascular and lymphatic plexus in the subintima secrete synovial fluid. These are found in synovial joints, bursae, and tendon sheaths. The joint cavity is lined with a synovial membrane. This membrane secretes synovial fluid that acts as a joint lubricant (Ombregt et al. 2003). Synovial sPLA₂ is isolated from this synovial fluid, for example mammalian Group IIA PLA₂. Other secreted PLA₂ (sPLA₂) is a phospholipase A₂ enzyme that is expressed in an extracellular secretion, for example, mammalian pancreatic sPLA₂, (e.g. human, bovine, rat, canine), mammalian synovial sPLA₂, and venom sPLA₂ (e.g. from bee, snakes, crotalids and elapids). In particular, secreted PLA₂ may be from venom from snake venom (Daboia russelli russelli).

Cytoplasmic or cytosolic PLA₂ (cPLA₂) is a phospholipase A₂ enzyme that is expressed in the cytoplasm, for example from neutrophils and platelets. Cytosolic PLA₂ has been shown to be important for macrophage production of inflammatory mediators, fertility, and in the pathophysiology of neuronal death after local cerebral ischemia (Bonventre et al., 1997).

The phospholipase may be a neutral venom phospholipase or an acidic venom phospholipase. Accordingly, phospholipase from neutral venom is referred to as neutral phospholipase, while phospholipase from acidic venom is referred to as acidic phospholipase. For example, an acidic PLA₂ isozyme is characterized as having at least one neutral or basic amino acid substituted with an acidic amino acid.

For example, a neutral venom phospholipase A₂ is characterized as having no overall acidic or basic charge. A basic venom phospholipase A₂ is characterized as having an overall basic charge. A basic venom phospholipase is not as favourable for clinical use because the basicity of PLA₂s is found to be usually correlated with their toxicity. It appears that the positively charged residues increase the enzyme penetrability into the membranes which is important for further hydrolysis of phospholipids and, thus, for the pharmacological potency (Kini, 1997).

According to a further aspect, the method of any aspect of the present invention may comprise the step of administering the phospholipase, isoform, derivative, mutant and/or fragment thereof:
at least once and/or continuously before the onset of the condition;
at least once and/or continuously during the onset of the condition; and/or
at least once and/or continuously after the onset of the condition.

The method according to any aspect of the present invention may comprise the step of administering phospholipase, isoform, derivative, mutant and/or fragment thereof, at least once and/or continuously, during and/or until about 24 hours after the onset of the condition; at least once and/or continuously during and/or until about 18 hours after the onset of the condition; for example, at least once and/or continuously during and/or until about 8 hours after the onset of the condition; at least once and/or continuously during and/or until about 4 hours after the onset of the condition; at least once and/or continuously during and/or until about 1 hour after the onset of the condition; at least once and/or continuously during and/or until about 30 minutes after the onset of the condition.

The method according to any aspect of the present invention may also comprise the step of administering the at least one phospholipase, isoform, derivative, mutant and/or fragment thereof, at 30 minutes, at around 1 hour, at around 4 hours, at around 8 hours, at around 10 hours, at around 18 hours and at around 24 hours or more, continuously or at any instant of time in between 30 minutes and around 24 hours. The dose at which the at least one phospholipase, isoform, derivative, mutant and/or fragment thereof is administered may be as indicated by the MIC, described in the examples below.

According to any aspect of the present invention, the methods may further comprise administering the at least one phospholipase, isoform, derivative, mutant and/or fragment thereof in conjunction with at least one pharmaceutically acceptable excipient, diluent, carrier and/or adjuvant.

According to another aspect, the present invention provides a pharmaceutical composition formulated for the treatment and/or prevention of at least one bacterial related condition. The composition may comprise a therapeutically effective amount of at least one phospholipase, isoform, derivative, mutant and/or fragment thereof. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. In particular, the related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli, with the proviso that the condition is not induced by Staphylococcus aureus and/or Escherichia coli when the phospholipase, isoform, derivative, mutant and/or fragment thereof is myotoxin II or BnpTx I. In particular, the condition is induced by Burkholderia pseudomallei (KHW) or Burkholderia pseudomallei (TES).

The at least one phospholipase, isoform, derivative, mutant and/or fragment thereof may be any suitable phospholipase, isoform, derivative, mutant and/or fragment thereof, as described above.

The present invention also provides a pharmaceutical composition formulated for the treatment and/or prevention of at least one bacterial related condition, wherein the pharmaceutical composition comprises a therapeutically effective amount of at least one phospholipase, isoform, derivative, mutant and/or fragment thereof comprising the amino acid sequence of SEQ ID NO:1 or SEQ ID NO:2. The bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. In particular, the condition is induced by Burkholderia pseudomallei (KHW) or Burkholderia pseudomallei (TES).

The pharmaceutical composition according to any aspect of the present invention may further comprise at least one pharmaceutically acceptable carrier, diluent, adjuvant, excipients, or a combination thereof. Examples of suitable excipients are water, saline, dextrose, glycerol, ethanol and the like as well as combinations thereof. Such a pharmaceutical composition may consist of the active ingredient alone, in a form suitable for administration to a subject, or alternatively the pharmaceutical composition may comprise the active ingredient and one or more pharmaceutically acceptable carrier, excipient and/or diluent. Excipients normally employed for such formulations, includes mannitol, lactose, starch, magnesium stearate, sodium saccharine, cellulose, magnesium carbonate, and the like.

The pharmaceutical composition according to any aspect of the present invention may be for local, subcutaneous, intravenal, injection, parenteral and/or oral administration. The pharmaceutical composition may be administered through subcutaneous and/or intramuscular injection. For oral administration, the pharmaceutical composition may be formulated as solutions, suspensions, emulsions, tablets, pills, capsules, sustained release formulations, aerosols, powders, or granulates. The dosage required depends on the choice of the route of administration; the nature of the formulation; the nature of the subject's illness; the subject's size, weight, surface area, age, and sex; other drugs being administered; and the judgment of the attending physician. Wide variations in the needed dosage are to be expected in view of the variety of compounds available and the different efficiencies of various routes of administration.

Another aspect of the present invention is a kit for treatment and/or prevention of at least one bacterial related condition, wherein the kit comprises a therapeutically effective amount of at least one phospholipase, isoform, derivative, mutant and/or fragment thereof. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. In particular, the related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli, with the proviso that the condition is not induced by Staphylococcus aureus and/or Escherichia coli when the phospholipase, isoform, derivative, mutant and/or fragment thereof is myotoxin II or BnpTx I. In particular, the condition is induced by Burkholderia pseudomallei (KHW) or Burkholderia pseudomallei (TES).

The at least one phospholipase, isoform, derivative, mutant and/or fragment thereof may be any suitable phospholipase, isoform, derivative, mutant and/or fragment thereof, as described above.

The present invention also provides a kit for the treatment and/or prevention of at least one bacterial related condition, wherein the kit comprises a therapeutically effective amount of at least one phospholipase, isoform, derivative, mutant and/or fragment thereof comprising the amino acid sequence of SEQ ID NO:1 and/or SEQ ID NO:2. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. In particular, the condition is caused by Burkholderia pseudomallei (KHW) or Burkholderia pseudomallei (TES).

The kit according to any aspect of the present invention may further comprise at least one pharmaceutically acceptable excipient, diluent, carrier and/or adjuvant, as described above. The kit may further comprise information and/or illustration for the use of the kit.

Another aspect of the present invention is an isolated peptide comprising at least one amino acid sequence selected from the group consisting of: SEQ ID NO:1, SEQ ID NO:2, an isoform, derivative, mutant and/or fragment thereof. The present invention also provides an isolated peptide having at least one amino acid sequence selected from the group consisting of: SEQ ID NO:1, SEQ ID NO:2, an isoform, derivative, mutant and/or fragment thereof. For example, the peptide of the present invention may be administered to a subject in the treatment and/or prevention of a bacterial related condition. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. For example, the condition may be melioidosis, septic shock and/or inflammation. In particular, the condition is induced by Burkholderia pseudomallei (KHW) or Burkholderia pseudomallei (TES).

The present invention also provides nucleic acid molecules encoding a peptide comprising at least one amino acid sequence selected from the group consisting of: SEQ ID NO: 1, SEQ ID NO:2, an isoform, derivative, mutant and/or fragment thereof. Also provided are nucleic acid molecules encoding a peptide having at least one amino acid sequence selected from the group consisting of: SEQ ID NO:1, SEQ ID NO:2, an isoform, derivative, mutant and/or fragment thereof. Nucleic acid molecules which hybridise to a nucleic acid molecule complementary to any nucleic acid molecule of the present invention or fragment thereof are also within the scope of the present invention.

A nucleic acid according to any aspect of the present invention may be a single or double stranded oligonucleotide or polypeptide, or can also be single or double stranded genomic DNA, cDNA, RNA, mRNA. In the case in which a nucleic acid molecule is transcribed and translated to produce a functional peptide, one of skill in the art recognizes that because of codon degeneracy a number of different nucleic acids encode the same peptide. In addition, the invention includes those peptides, referred to as isoforms, derivatives or mutants of the peptide comprising the sequence of SEQ ID NO:1 and/or SEQ ID NO:2, that comprises amino acid sequence substantially identical with the peptide comprising the sequence of SEQ ID NO:1 and/or SEQ ID NO:2 for administering to a subject in the treatment and/or prevention of a bacterial related condition. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. For example, the condition may be melioidosis, septic shock and/or inflammation. In particular, the condition is induced by Burkholderia pseudomallei (KHW) or Burkholderia pseudomallei (TES).

Isoforms, derivatives and/or mutants include peptides with conserved amino acid changes in the sequence comprising SEQ ID NO:1 and/or SEQ ID NO:2. Conserved amino acid substitutions are those changes that can be made without altering the biological function of the native peptide comprising the sequence of SEQ ID NO:1 and/or SEQ ID NO:2. In particular, variants of the peptide comprising the sequence of SEQ ID NO:1 and/or SEQ ID NO:2, may be defined as those peptides that contain amino acid substitutions, wherein an amino acid can be replaced with another amino acid without altering the biological activity of the peptide. These amino acids may or may not be conserved across species and may or may not be essential to the biological activity of the peptide.

The present invention also encompasses homologues of the peptide comprising the sequence of SEQ ID NO:1 and/or SEQ ID NO:2 that are “substantially identical” to the peptide comprising the sequence of SEQ ID NO:1 and/or SEQ ID NO:2, which retain the biological activity of the peptides according to any aspect of the present invention. Homologues of the peptide comprising the sequence of SEQ ID NO:1 and/or SEQ ID NO:2 are defined by their ability in the treatment and/or prevention of a bacterial related condition. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. For example, the condition may be melioidosis, septic shock and/or inflammation. In particular, the condition is induced by Burkholderia pseudomallei (KHW) or Burkholderia pseudomallei (TES). The critical amino acids in a protein or peptide are those amino acids that are important for the function of the protein or peptide. These amino acids are often conserved across different species and organisms. It is well known to one skilled in the art that conserved amino acids are usually identical across species and organisms and if not, are very similar in their chemical properties.

A nucleic acid molecule is “hybridisable” to another nucleic acid molecule (in the present case, a nucleic acid molecule complementary to the nucleic acid molecule encoding a peptide comprising at least one amino acid sequence selected from the group consisting of: SEQ ID NO:1, SEQ ID NO:2, an isoform, derivative, mutant and/or fragment thereof, when a single-stranded form of the nucleic acid molecule can anneal to the other nucleic acid molecule under appropriate conditions of temperature and solution ionic strength (Sambrook and Russell, Molecular Cloning, A Laboratory Manual, Cold Spring Harbour Laboratory Press, 2001). The conditions of temperature and ionic strength determine the “stringency” of the hybridisation. Hybridisation requires the two nucleic acids to contain complementary sequences. Depending on the stringency of the hybridisation, mismatches between bases are possible. The appropriate stringency for hybridising nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridisation decreases in the following order: RNA:RNA, DNA:RNA, DNA:DNA. For hybrids of greater than 100 nucleotides in length, equations for calculating Tm have been derived (Sambrook and Russell, 2001, as above). For hybridisation with shorter nucleic acids, i.e. oligonucleotides, the position of mismatches becomes more important, and the length of the oligonucleotide determines its specificity (Sambrook and Russell, 2001, as above). Preferably a minimum length for a hybridisable nucleic acid is at least about 10 nucleotides; more preferably at least about 15 nucleotides; most preferably the length is at least about 18 nucleotides.

The present invention also provides a vector comprising at least one of the nucleic acid molecules described above. The vector may further comprise a regulatory nucleic acid sequence linked to the nucleic acid molecule encoding the polypeptide comprising the sequence of SEQ ID NO:1 and/or SEQ ID NO:2. The regulatory nucleic acid may be a prokaryotic or eukaryotic promoter.

The invention further provides a host cell comprising the vector according to any aspect of the present invention. The host cell may be present in the form of a cell culture. The host cell may be a prokaryotic or eukaryotic cell.

In particular, the host cell is cultured to express a peptide comprising at least one amino acid sequence selected from the group consisting of: SEQ ID NO:1, SEQ ID NO:2, an isoform, derivative, mutant and/or fragment thereof. The peptide may be administered to a subject in treatment and/or prevention of a bacterial related condition. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. For example, the condition may be melioidosis, septic shock and/or inflammation. In particular, the condition is induced by Burkholderia pseudomallei (KHW) or Burkholderia pseudomallei (TES). The host cell may also express at least a peptide having at least one amino acid sequence selected from the group consisting of: SEQ ID NO:1, SEQ ID NO:2, an isoform, derivative, mutant and/or fragment thereof.

Methods for the preparation of a vector comprising a nucleic acid molecule according to the invention, preferably linked to a promoter, and cultivation of the host cell comprising the vector can be carried out according to standard techniques, such as those indicated or described in Sambrook and Russel, Molecular Cloning, Cold Spring Harbour, 2002.

The peptide according to any aspect of the present invention may be a fused peptide and comprise at least one peptide comprising the sequence of SEQ ID NO:1 and/or SEQ ID NO:2.

The peptide according to any aspect of the invention may be isolated and/or purified from biological material, expressed from recombinant DNA, and/or prepared by chemical synthesis. In particular, the peptide may be isolated and/or purified from a human or non-human animal species.

For example, the peptide of the invention is isolated and/or purified from venom. The peptide may be isolated and/or purified according to any standard technology known in the art. In particular, the peptide may be obtained by steps comprising:
obtaining crude venom;
carrying out gel filtration; and
performing reverse-phase high performance liquid chromatography.

However, any other suitable method of isolating and/or obtaining the peptide according to any aspect of the invention known in art may be used.

According to a particular aspect, the peptide according to any aspect of the invention may be isolated and/or purified from the venom of Daboia russelli russelli. Accordingly, the peptide of the invention may be isolated and/or purified by the steps comprising:
obtaining crude venom of Daboia russelli russelli;
carrying out gel filtration; and
performing reverse-phase high performance liquid chromatography.

The peptides isolated form the crude venom of Daboia russelli russelli is indicated as DRR1-PLA₂ and DRR2-PLA₂ protein or peptide.

In particular, the gel filtration step may be a sequential gel filtration. However, the method for isolation and/or purification of peptides according to the invention, in particular the peptides DRR1-PLA₂ and DRR2-PLA₂, is not limited to that exemplified herein. Any suitable method known in the art may also be used. The primary structure of DRR1-PLA₂ and DRR2-PLA₂ respectively is as follows:

| | |
|---|---|
| SLLGFGCMILEETGVMIELEKNCNQHPE; | (SEQ ID NO:1) |
| SLLEFGMMILEETGKLAVPFYSKYGLYCGCGGKTPDD | (SEQ ID NO:2) |

In particular, the peptides of the present invention have a molecular weight of 13822 Da or 13669 Da.

The present invention also provides isolated and/or purified venom comprising peptides for treatment and/or prevention of a bacterial related condition. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. In particular, the condition is induced by Burkholderia pseudomallei (KHW) or Burkholderia pseudomallei (TES).

According to another aspect, the present invention provides a pharmaceutical composition comprising a therapeutically effective amount of isolated and/or purified venom for treatment and/or prevention of a bacterial related condition. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. For example, the condition may be melioidosis, septic shock and/or inflammation. In particular, the condition is induced by Burkholderia pseudomallei (KHW) or Burkholderia pseudomallei (TES).

According to any aspect of the present invention, the at least one isolated and/or purified venom may be selected from the group consisting of: Daboia russelli russelli, Daboia russelli siamensis, Daboia russelli pulchella, Crotalus adamanteus, Crotalus durissus terrificus, Pseudechis australis, Agkistrodon halys, Pseudechis guttata, Bitis arietans, Bitis gabonica rhinoceros, Echis carinatus, Acanthopis antarticus, Bungarus candidus, Bothrops asper, Bothrops jararacussu and/or Apis mellifera.

The present invention also provides a method for the treatment and/or prevention of a bacterial related condition comprising administering to a subject a therapeutically effective amount of at least one isolated and/or purified venom selected from the group consisting of: Daboia russelli russelli, Daboia russelli siamensis, Daboia russelli pulchella, Crotalus adamanteus, Crotalus durissus terrificus, Pseudechis australis, Agkistrodon halys, Pseudechis guttata, Bitis arietans, Bitis gabonica rhinoceros, Echis carinatus, Acanthopis antarticus, Bungarus candidus, Bothrops asper, Bothrops jararacussu and/or Apis mellifera. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. For example, the condition may be melioidosis, septic shock and/or inflammation. In particular, the condition is induced by Burkholderia pseudomallei (KHW) or Burkholderia pseudomallei (TES).

The present invention also provides a medicament for the treatment and/or prevention of a bacterial related condition comprising administering to a subject a therapeutically effective amount of at least one isolated and/or purified venom selected from the group consisting of: Daboia russelli russelli, Daboia russelli siamensis, Daboia russelli pulchella, Crotalus adamanteus, Crotalus durissus terrificus, Pseudechis australis, Agkistrodon halys, Pseudechis guttata, Bitis arietans, Bitis gabonica rhinoceros, Echis carinatus, Acanthopis antarticus, Bungarus candidus, Bothrops asper, Bothrops jararacussu and/or Apis mellifera. For example, the bacterial related condition may comprise at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli. For example, the condition may be melioidosis, septic shock and/or inflammation. In particular, the condition is induced by Burkholderia pseudomallei (KHW) or Burkholderia pseudomallei (TES).

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

### Example 1

Methods

Venoms

Lyophilized venoms—Acanthophis augtra, Acanthophis antarcticus, Acanthophis praelongus, Acanthophis Pyrrhus, Androctonus australis, Bungarus candidus, Hyrophis cyanocinctus, Naja naja naja, Notechis aterater, Naja sumatrana, Naja kaouthia, Pseudechis australis, Pseudechis guttata, Pseudechis porphyriacus, Pseudechis colletti, Pseudonaja inframaggula, Pseudonaja nuchalis, Pseudonaja textilis, Pseudechis affinis, Rhabdophis tigrinus, Oxyuranus scutellatus, Agkistrodon halys, Bitis gabonica rhinoceros, Crotalus adamanteus, Echis carinatus, Daboia russelli russelli, Daboia russelli siamensis, Trimeresurus wagleri, Apis mellifera, Bothotus hottenlota, Buthotus hottenota hottenota, Buthus martensii and Naja naja naja venoms were extracted from long-term captive specimens. Venoms from captive specimens were collected manually by milking. Each sample of freeze-dried venom was stored at 4° C. Lyophilized venom of D. russelli russelli (Indian) was purchased from commercial sources (Venom Supplies Pte Ltd, Tanunda, South Australia). The venom samples were collected using a 50 ml capillary tube placed over the enlarged rear maxillary fangs to minimize contamination by saliva. The venom samples were collected in a sterile manner under strict laboratory conditions, and were transferred to microcentrifuge tubes, immediately frozen, lyophilized and stored at 220.8° C. until used. The dried venom was normally packed and stored in the dark at −20° C. The solid venoms were obtained from commercial sources (Venom supplies Pte Ltd, Tanunda, South Australia). L-amino acid oxidase purified from the venom of B. atrox and C. adamanteus were obtained commercially (Sigma Aldrich, St Louis, Mo., USA).

Purification of Phospholipase A₂ (PLA₂) Enzymes

Phospholipase A₂ enzymes were purified from their corresponding crude venoms as described (Thwin M M et al, 1995) with minor modification. All steps for the fractionation of the lyophilized crude venom of D. russelli russelli was carried out at room temperature. About 500 mg of dried whole venom was extracted with 10 ml of 50 mM (pH 7.4) Tris-hydrochloric acid buffer and the suspension was centrifuged at 500 g at 4° C. for 10 min and filtration with a 0.22 mm syringe filter to remove any colloidal or particulate material. Aliquots of the yellowish clear supernatant was loaded on a Sephadex G-75 column (1.6×40 cm; Amersham Pharmacia (GE Healthcare, Sweden)) previously equilibrated with the same buffer (50 mM Tris-HCl, pH 7.4). Fractions (2 ml) were collected at a flow rate of 15 ml/hr. The absorbance of all the fractions was monitored at 280 nm. Five fractions (F1, F2, F3, F4, and F5) were collected from the single pool of venom fractionated on G-75 column and aliquots taken for testing antibacterial, enzymatic activities and for protein measurement. The fraction with highest antibacterial and PLA₂ activity was further purified by reverse-phase (RP-HPLC) on C18 and C8 columns (Jupitor Phenomex) in 0.1% trifluoroacetic acid (TFA) eluted with a linear gradient of 80% acetonitrile (ACN) in 0.1% TFA. Protein fractions were collected with a FC 905 B fraction collector (0.5 min) and related fractions (RV5 & RV6) were pooled separately for further analysis. Elution of proteins was monitored at 280 nm and 215 nm. The homogeneity of purified PLA₂s was determined by using MALDI-TOF on a Voyager DE-STR Biospectrometry workstation (Applied Biosystem CA, USA).

Protein Assay

The protein concentration of the crude venom solutions was determined using the Bio-Rad protein assay reagent (Bradford M M, 1976) and bovine serum albumin as a standard.

Protein Analysis and SDS-PAGE

The purity of isolated DRR1-PLA₂ and DRR2-PLA₂ were verified using (14% acrylamide Trisglycine) by sodium dodecylsulphate polyacrylamide gel electrophoresis (SDS-PAGE) according to Laemmli (1970). Separating gels containing 15% acrylamide and stacking gels of 4.5% acrylamide were used. The fractions were diluted with 1:1 with sample buffer (0.12 M Tris-HCl, pH 6.8 containing 2% SDS, 5% 2-mercapethanol, 10% glycerol, 0.02% bromophenol blue) and heated for 5 min in a boiling water bath. Electrophoresis was carried out at a constant current 20 mA for 2.5 h. The gel was fixed with 5% acetic acid overnight and stained for 2 h in 0.1% Coomassie blue R-250 in 5% acetic acid. Destaining was carried out in a solution containing 35% methanol and 7% acetic acid until the background become clear. The molecular weights of protein bands were determined using Bio-Rad SDS mol. weight standard markers.

Mass Determination by MALDI TOF-TOF

Analyses were performed primarily using a Perspective Biosystem matrix-assisted laser desorption ionization-time of flight (MALDI-TOF) Voyager-DE mass spectrometer (Framingham, Mass.), operated in delayed extraction mode. The enzymes were analyzed using a saturated solution of -cyano-4-hydroxycinnamic acid (Sigma) in acetone containing 1% trifluoroacetic acid (Sigma). The PLA₂s were selected in the mass range of 1000-30000 kDa. Spectra were calibrated using calibration mixture 2 of the Sequazyme peptide mass standards kit (Perspective Biosystems, Framingham, Mass.). The search program MS-Fit, was used for searches in the database NCBI. MALDI-TOF mass spectrometry was used for molecular weight determination.

N-Terminal Sequencing

Suitable enzymes were subject to N-terminal sequencing by Edman degradation using an Applied Biosystems 494 pulsed liquid-phase sequencer equipped with an on-line 120 A PTH-amino acid analyzer. The resulting amino acid sequences were subjected to protein-protein BLAST (Basic Local Alignment Search Tool). When the N-terminal sequence was searched for similarity, the DRR1-PLA₂ and DRR2-PLA₂ were not matching in the existing data and DRR1-PLA₂ & DRR2-PLA₂ masses are different.

Biochemical Characterization

Phospholipase A₂ Enzyme Activity

Phospholipase A₂ (PLA₂) catalyzes the hydrolysis of phospholipids at the sn-2 position yielding a free fatty acid and a lysophospholipid. The Cayman Chemical secretory PLA₂ (sPLA₂) assay kit was used for the measurement of enzyme activity. This assay uses the 1,2-dithio analog of diheptanoyl phosphatidylcholine which serves as a substrate for most PLA₂s (Reynolds L J et al, 1992). sPLA₂ specific activity is expressed as μmole/min/mg protein. The PLA₂ enzyme activity is also converted to μ moles of fatty acid released per min per mg phospholipase by the decrease in absorbance produced by known amount of acid. A decrease in absorbance of 0.1 was obtained with 0.025μ moles of HCl in the reaction mixture with the present method.

Antimicrobial Activity

Six clinical isolates of Gram-negative bacteria Escherichia coli, Enterobacter aerogenes, Proteus vulgaris, Proteus mirabilis, Pseudomonas aeruginosa, Burkholderia pseudomallei (TES & KHW) and Gram-positive bacteria Staphylococcus aureus were obtained from the Department of Microbiology, Yong Loo Lin School of Medicine, National University of Singapore. The following antimicrobial agents: Chloramphenicol 30 μg/disc and Ceftazidime 30 μg/disc (Becton Dickinson Labware, USA) were included as positive controls and blank discs with sterile double distilled water served as negative control. Each sample of freeze-dried venom was dissolved in 1 mL of 50 mM Tris-HCl buffer (pH 7.4), vortexed (Labnet VX100), and filtered using 0.22 mμ syringe filter (Millipore, N.Y., USA) before storage at 4° C. All the venoms were tested for their antibacterial activity by disc-diffusion susceptibility tests performed following standards recommended by the NCCLS (National Committee for Clinical Laboratory Standards, M2-A6, 1997) with some modifications. The bacterial cultures were spread and allowed to grow overnight at 37° C. on 20 ml Mueller Hinton agar (Oxoids Pte Lte, UK) (pH 7.4) or TS agar plates (90 mm diameter). The surface of the medium was allowed to dry for about 3 min. Antimicrobial susceptibility was tested according to the method described by Bauer et al., 1966. Sterile paper discs (7 mm diameter) were then placed onto the surface of the plate and 20 μL (0.1 mg/mL) of venom sample was added per disc in 5 replicates. Disc containing 20 μL of Tris-HCl buffer served as a normal control and discs containing antibiotics were used as drug controls. The plates were incubated at 37° C. for 24 h, following which the diameter of inhibition zones were measured as mm (inhibitory zones).

Antibacterial Effects of Purified PLA₂

All the venom enzymes used in the experiment have been purified by successive chromatographic steps with the final purity of at least 95% as assessed by Reversed-phase HPLC. The activities of the following phospholipase A₂ enzymes purified from the venoms of different snake species (in parenthesis)—crotoxin A (Crotalus durissus terrificus), crotoxin B (Crotalus durissus terrificus), ammodytoxin A (Vipera ammodytes ammodytes), mojavetoxin (Crotalus scutulatus scutulatus), β-bungarotoxin (Bungarus multicinctus), taipoxin (Oxyuranus scutellatus scutellatus), mulgatoxin (Pseudechis australis), Daboiatoxin (Daboia russelli russelli), honey bee (Apis mellifera) venom phospholipase A₂—and two L-amino acid oxidase—LAAO (B. atrox), LAAO (C. adamanteus) were evaluated. 0.5 μmoles of each of the enzymes dissolved in 500 μL of 50 mM Tris-HCl (pH 7.4) buffer was examined. In vitro antimicrobial activity was determined by the previously described disc-diffusion method (National Committee for Clinical Laboratory Standards, M2-A6, 1997) with some modifications.

MIC Determinations

In preliminary experiments, the disc-diffusion assay for determining antibacterial effects of crude venoms was compared to the activity of isolated phospholipase A₂ (PLA₂) enzymes using broth-microdilution method. Differences in data between these two assays were observed. Thus, we used the broth-microdilution technique to test the activities of the phospholipase A₂ against pathogen from patients by comparing their activities to the activity of the antibiotics. Bacteria from frozen suspensions were sub-cultured onto Tryptic soya (TS) agar plates and passaged twice prior to susceptibility testing. The bacteria were grown in Tryptic soy broth (TSB) for 5-7 h (exponential phase) before adjusting their concentration to a 0.5 McFarland turbidity standard. The adjusted bacterial cultures were diluted to approximately (A₆₀₀ of 0.8) 3.2×10⁸ colony forming units (cfu/mL). The enzymes (PLA_{2S}) to be examined were dissolved in 1 M Tris-HCl buffer (pH 7.4) for determination of the activities (MIC). The bacteria were washed and incubated with the enzymes in appropriate buffer. MICs were determined by the broth microdilution method recommended by the NCCLS (National Committee of Clinical Standards, 1997, M7-A4; Amsterdam D, 1996). Serial dilutions of isolated PLA₂ enzyme solutions (final concentration 0.5, 0.25, 0.125, 0.0625 and 0.03125 mg/mL) and DRR2-PLA2s solutions (final concentration 42 μM, 20.5 μM, 10.25 μM, 5.12 μM, and 2.56 μM) were prepared in microtiter trays with appropriate medium TSB and MH respectively. Each dilution series included control wells containing bacteria without enzymes. A total of 200 μL of the adjusted inoculum of 10⁵ cfu/mL was added to each well (96-well plates). The culture trays were incubated at 37° C. for 24 h with shaking at 230 rpm. The inhibition of bacterial growth was determined by measuring the absorbance at 560 nm (Molecular Devices E precision microplate reader). The MIC was taken as the lowest concentration of phospholipase A₂ or DRR2-PLA₂s enzyme that inhibited visible growth of the organism. The results given are mean values of three independent (n=3) determinations.

Hemolytic Assay

The hemolytic activity of the DRR1-PLA₂ & DRR2-PLA₂ was assayed with heparinized human red blood cells that had been collected from a normal volunteer and washed three times in 1× phosphate-buffered saline. A 10% suspension of red blood cells was combined with PLA₂s phosphate-buffered saline (negative control), and 10% Triton X-100 (positive control for 100% hemolysis) in a final volume 200 μl. After a 30 minutes incubation, cell suspensions were centrifuged for 10 min at 1,300× g and supernatants were transferred to a flat-bottom 96-well polystyrene microtiter plates and the absorbance (A) was read at 540 nm (E-max, MolecularDevice, Research Instrument, Singapore). The percentage of hemolysis was calculated using the formula 100×(Aₛₐₘₚₗₑ−A_{blank})/(A_{Triton}−A_{blank}) (Travis et al., 2000).

Scanning Electron Microscopy (SEM)

The structural changes induced by DRR2-PLA₂s peptides on S. aureus, P. vulgaris and P. mirabilis and Burkholderia pseudomallei (KHW) were studied using SEM as described in Motizuki et al., 1999. Each of the peptide 50 μl (42-2.56 μM) samples that contained (S. aureus, B. pseudomallei, P. vulgaris and P. mirabilis) 1 ml (3.2×106 CFU/ml.) in MH broth was preincubated for 30 min at 37° C. The control received equivalent volumes of MH broth containing bacteria with 100 μl calcium chloride (7.5 mM). After removing a small portion of these samples for CFU/ml measurements, the remainder was centrifuged for 10 min at 2,800 g. Pellets were resuspended and fixed with an equal volume of 2.5% glutaraldehyde in 1 mM phosphate buffer (pH 7.4) for 1 h in. Immediately following the addition of the fixative solution, the sample tubes were mixed by gently inverting the tube up and down for several minutes to prevent clumping of the cells. The cells postfixed for an additional hour with 1% Osmimum tetroxide (OsO₄) and washed thrice in PBS. 1 μl of samples were pipetted on sterile cover glass coated with Poly-L lysine and left to stand for 20-30 mins. The section was dehydrated by series of alcohol (25%, 50%, 75%, 90% 100%). The samples were transferred in 100% ethanol to a critical point dryer (Balzers CPD-030, Bal-Tec AG, Vaduz, Liechtenstein), and dried using liquid carbon dioxide. The samples were mounted on aluminum specimen supports with carbon adhesive tabs, and coated with a 10-15 nm thickness of gold using a sputter coater SC D005 (Bal-Tec, EM Technology and Application, Liechtenstein). Samples were examined with a Philips XL 30 FEG SEM (Electron Microscopy, Holland) using an accelerating voltage of 5-10 kV.

Transmission Electron Microscopy (TEM)

The structural changes induced by DRR2-PLA2 on S. aureus P. vulgaris, P. mirabilis and B. pseudomallei (KHW) were studied using Transmission electron microscopy as described earlier (Matsuzaki, 1998). Bacterial cells suspended in 10 mM phosphate buffer (pH 7.4) after treating with 5.12 μM of DRR2-PLA₂ were fixed with an equal volume of 2.5% glutaraldehyde in 10 mM phosphate buffer, pH 7.4. The fixed samples were stored overnight at 4° C. in the fixative solution. The suspended fixed cells were rinsed with 10 mM phosphate buffer, and dehydrated through a graded series of ethanols (25-100%). During the entire filtration, rinsing, and dehydration process, the cells were kept covered with fluid to prevent air drying. The samples were transferred in 100% ethanol to a critical point dryer (Balzers CPD-030, Bal-Tec AG, Vaduz, Liechtenstein), and dried using carbon dioxide. The samples were mounted on aluminum specimen supports with carbon adhesive tabs, and coated with a 15 nm thickness of goldpalladium metal (60:40 alloy) using a Hummer X sputter coater (Bal-Tec, EM Technology and Application, Liechtenstein). Samples were examined with a (JEF 2220) TEM using an accelerating voltage of 5-10 kV.

Cell Proliferation Assay (XTT-Based Cytotoxicity Assay)

The human macrophage cell line (U-937) and the monocytic human macrophage cells (THP-1) were purchased from ATCC (Virginia, USA) and cell Proliferation Kit II was from Roche Applied Sciences (Singapore). Sterile Roswell Park Memorial Institute (RPMI) medium and Dulbecco's Modified Eagle's Medium (DMEM), Fetal Bovine Serum (FBS), 1 M Tris-HCl buffer (pH 7.4), and 10 mM HEPES (ATCC, Virginia, USA) were purchased from National University Medical Institute (NUMI), Singapore. All chemicals were of analytical and cell culture grade. Human macrophage, U-937 cell line and THP-1 cell line were cultured in 72 cm² flasks at a density of 1×10⁷ cells/12 mL in RPMI and DMEM culture medium respectively, supplemented with 10% fetal bovine serum (FBS), and 1 ml of HEPES. The cell viability was measured using tetrazolium salts (XTT) as described (Langer et al, 2001).

Briefly, the cells were allowed to adhere to the bottom of the flask for overnight at 37° C. in a humidified atmosphere of 5% CO₂ and 95% air. The culture medium was changed three times a week. To analyze the initial events of venom-mediated cell viability, five different venoms (0.05-10 mg/mL) and DRR2-PLA₂ (0.1-2.5 mM) were applied to cultured macrophage cell lines at varied time intervals (12, 24, and 48 h). Cell proliferation was spectrophotometrically quantified using an ELISA plate reader at 490 nm. All assays were prepared in triplicates and repeated thrice. The cytotoxicity of purified PLA₂ enzymes was also tested at different concentrations (0.05-10 μg/mL) using XTT assay.

Invasion Cytotoxicity by Lactate Dehydrogenase (LDH)

Cytotoxic effects of bacteria on human macrophages cells were evaluated by measuring the release of lactate dehydrogenase (LDH) enzyme using a cytotoxicity detection kit (Roche Mannheim, Germany). Mid-log phase bacterial cells (3.5×105) were added to THP-1 cells (1×106 cells/well) cultured on 96-well plates in DMEM medium (NUMI, Singapore) supplemented with 10% (vol/vol) fetal bovine serum (Sigma). The multiplicity of infection was 100 percent, after 1 h incubation at 37° C. in the presence of 5% CO2. DRR2-PLA₂ (0.1-2.5 mM) was added and the cells were further incubated for 24 h and 48 h. A 200 μl aliquot of the centrifuged supernatant obtained from each well was used for the quantification of cell death and cell lysis, based on the measurement of lactate dehydrogenase (LDH) activity released from the cytosol of damaged cells into the supernatant. The assay was performed in triplicate (Elsinghorst, 1994).

Statistical Analysis

The results (mean±S.D, n=5) were analysed by one way-ANOVA with repeated measures to analyze factors influencing the zone size of growth inhibition, and for comparison with standard drugs.

Results and Discussion

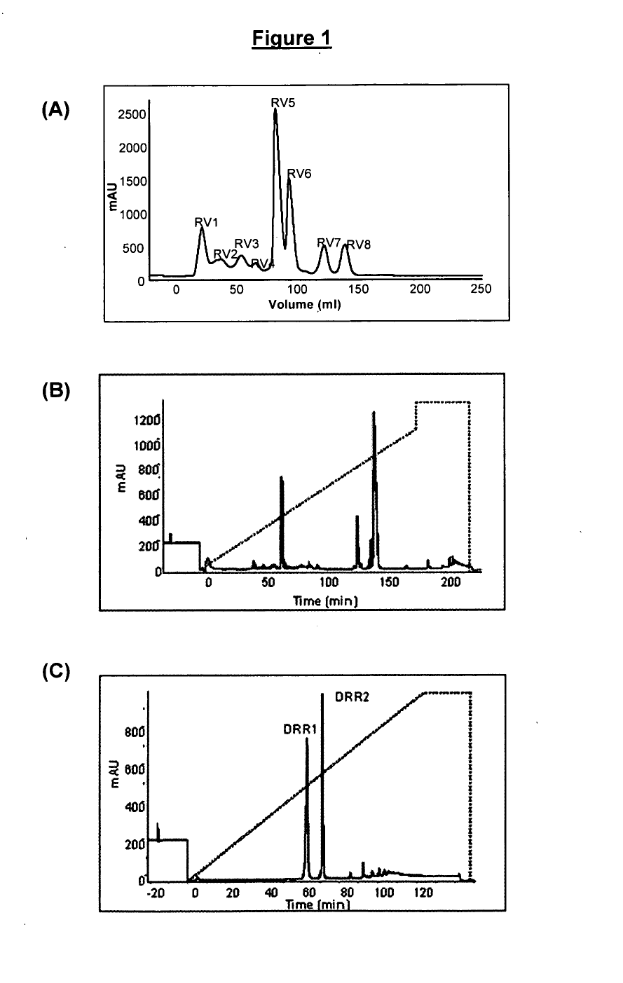

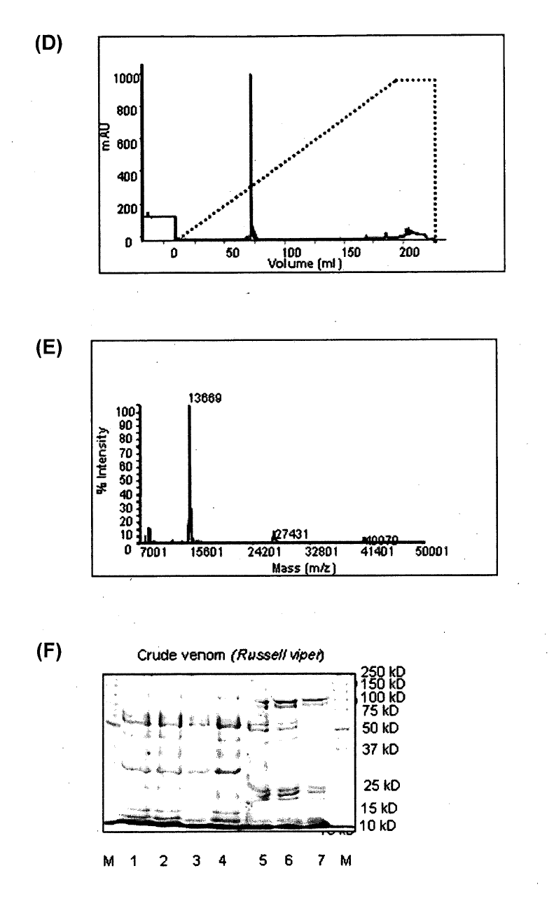

Russell's viper (Daboia russelli ressulli) venom purified by using the following gel filtration chromatography on a Superdex G-75 column produced eight major protein peaks as shown in (FIG. 1A). All the fractions (RV1 to RV8) were assayed for antibacterial activity, among which fraction RV6 showed significant antibacterial and phospholipase A₂ activity than fraction RV5. The active fraction RV6 was further fractionated by reverse phase chromatography on Sepharose (C18 column), and resolved into four further fractions namely RV-D1-RV-D4 (FIG. 1B). The most active antibacterial fraction RV-D1 subjected to applied on Sepharose C18 and C8 reverse phase chromatography, resolved into two major purified proteins (FIG. 1C), and designated Daboia russelli ressulli-phospholipase A₂ (DRR1-PLA₂ and DRR2-PLA₂). The homogenecity of PLA₂s were assessed by SDS-PAGE, and its molecular masses were estimated to be approximately 15 kDa (FIG. 1H). Homogeneity of the DRR1-PLA₂ & DRR2-PLA₂ were further demonstrated by native PAGE, digestion, gel spot and BLAST search was not matched with previously reported PLA₂s of Viperidae groups. However, the MALDI-TOF/MS analysis showed the actual mass of protein DRR1-PLA₂ (Molecular weight 13822.44) and DRR2-PLA₂ (Molecular weight 13669.93). The final protein concentration was quantified. The N-terminal amino acid residues of DRR1-PLA₂ and DRR2-PLA₂ were sequenced, and compared with the sequences in the EXPASY proteomics database using BLAST search (Tables 1a & b). The amino acid sequences were not matched exactly with the available sequences and its protein masses varied from the existing phospholipase A₂ₛ.

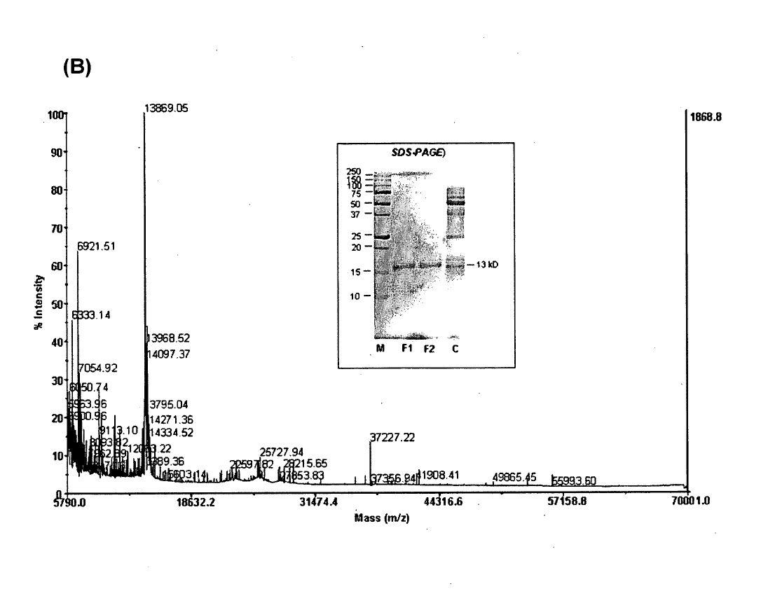

The purified PLA₂ enzyme of A. halys was also subjected to purification followed by MALDI-TOF analysis as shown in FIG. 23. The MALDI-TOF/MS analysis showed the actual mass of proteins were 23146.61 and 13869.05.

TABLE 1 (a)
N-terminal aminoacid sequences of DRR2-PLA₂ from the venom of D. russelli russelli. The molecular
masses of phospholipase A_{2 enzyme did not match with reported} Daboia species. The folded residues
different from those of the reported are shown with masses.

| SN | Accession No | Amino acid sequences | MW |
|---|---|---|---|
| | DRR2-PLA2 | 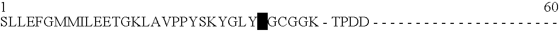 | 13669.93 |
| 1 | gi\|31615954 | 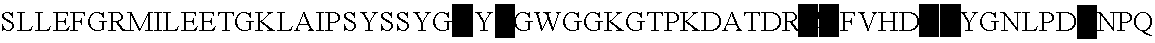 | 14428 |
| 2 | gi!49259307 | 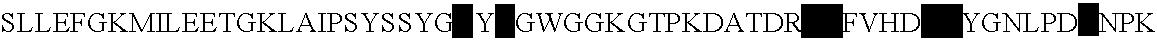 | 14400 |
| 3 | gi\|31615955 | 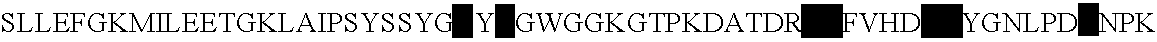 | 14428 |
| 4 | gi\|50513756 | 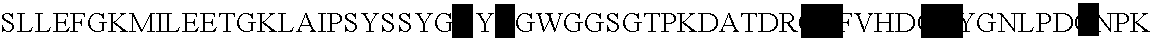 | 14359 |
| 5 | gi\|48425253 | 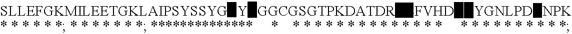 | 14306 |
| SN | Accession No | Amino acid sequences | MW |
| | DRR2-PLA2 | 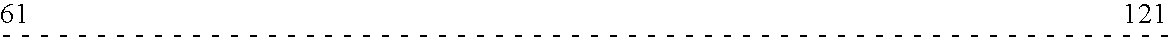 | 13669.93 |
| 1 | gi\|31615954 | 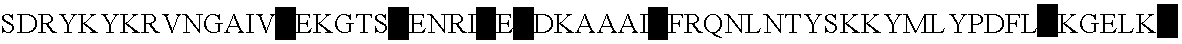 | 14428 |
| 2 | gi!49259307 | 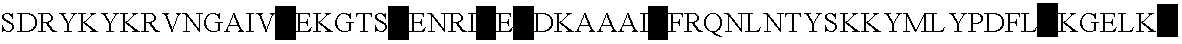 | 14400 |
| 3 | gi\|31615955 | 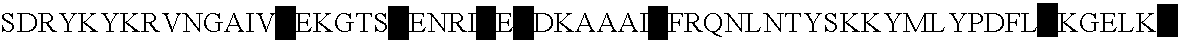 | 14428 |
| 4 | gi\|50513756 | 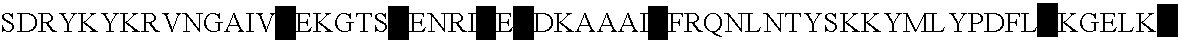 | 14359 |
| 5 | gi\|48425253 | 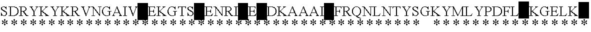 | 14306 |

TABLE 1 (b)
N-terminal aminoacid sequences of DRR1-PLA₂ from the venom of D. russelli russelli. The molecular
masses of phospholipase A₂ enzyme did not match with reported Daboja species. The folded residues
different from those of the reported are shown with masses.

| SN | Accession No | Amino acid sequences | MW |
|---|---|---|---|
| 1 | DRR1-PLA2 | 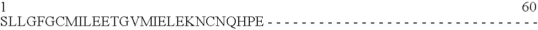 | 13,822.44 |
| 2 | gi\|31615954 | 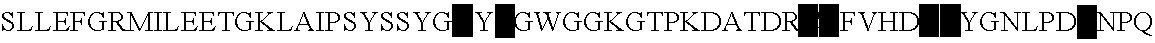 | 14428 |
| 3 | gi!49259307 | 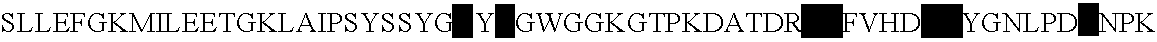 | 14400 |
| 4 | gi═31615955 | 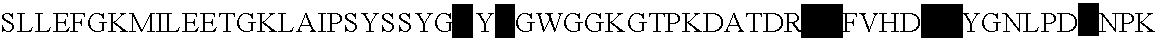 | 14428 |
| 5 | gi═50513756 | 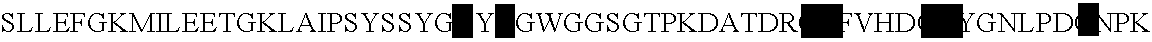 | 14359 |
| | gi═48425253 | 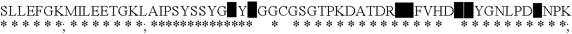 | 14306 |
| SN | Accession No | Amino acid sequences | MW |
| 1 | DRR1-PLA2 | 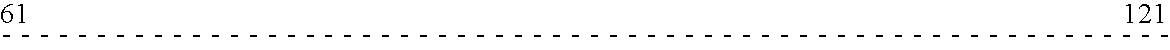 | 13,822.44 |
| 2 | gi═31615954 | 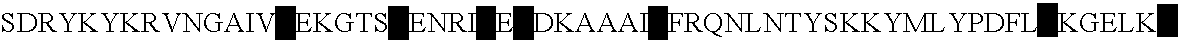 | 14428 |
| 3 | gi!49259307 | 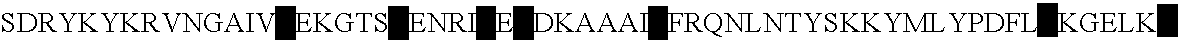 | 14400 |
| 4 | gi\|31615955 | 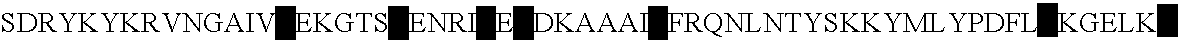 | 14428 |
| 5 | gi\|50513756 | 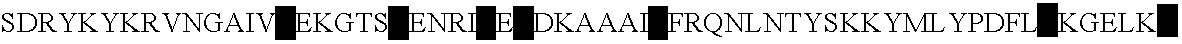 | 14359 |
| | gi\|48425253 | 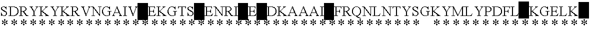 | 14306 |

Antimicrobial Activity

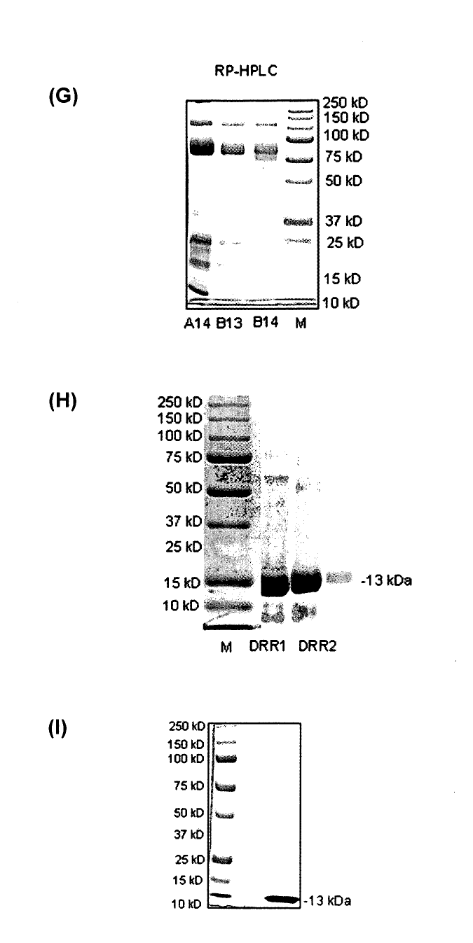

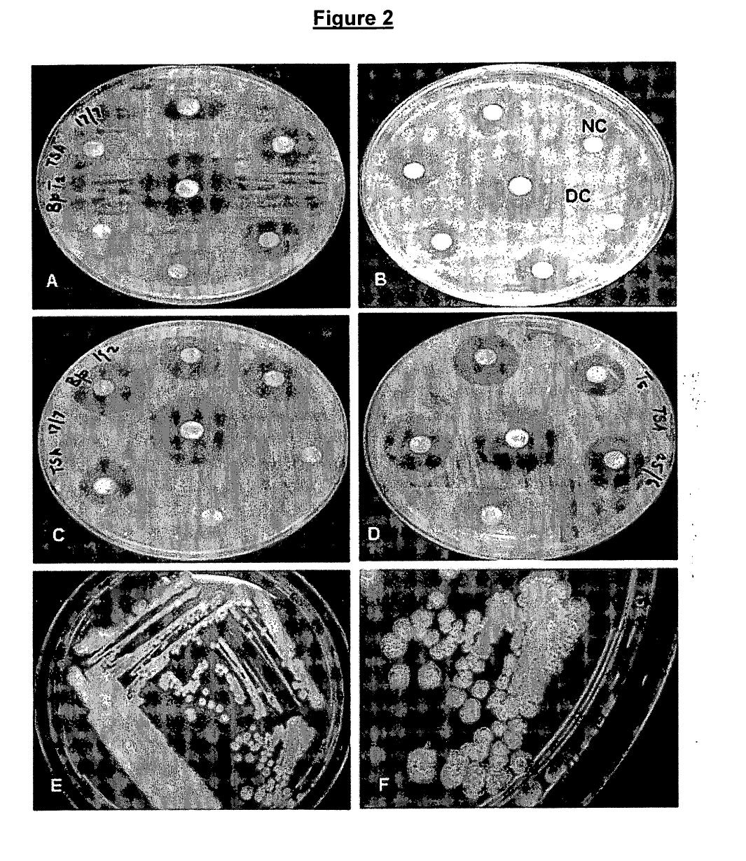

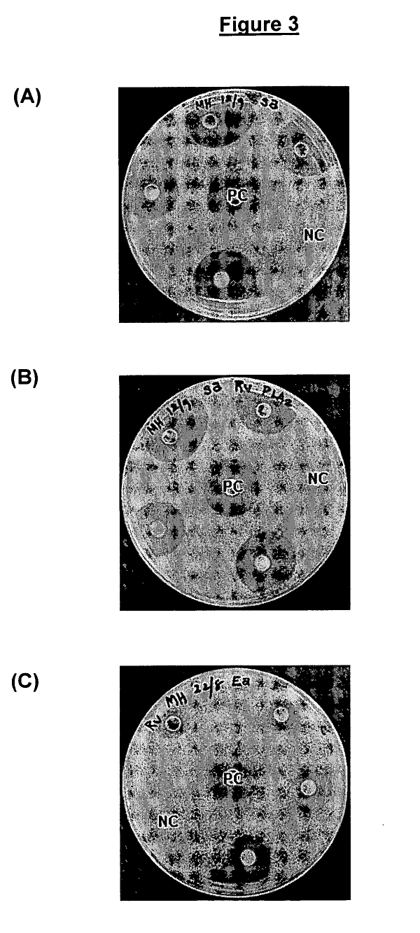

Purified phospholipase A₂ enzymes were tested their antibacterial properties against potent Gram-positive and negative bacteria at 84 μM concentration for DRR1-PLA₂ and DRR2-PLA₂. The enzymes exhibited broad spectrum of antimicrobial activity against a wide range of pathogenic organisms. In the present study, the bacterial strains B. pseudomallei (KHW & TES), S. aureus, E. aerogenes, P. vulgaris, P. mirabilis, P. aeruginosa, E. coli proved to be highly or intermediately susceptible to various snake venoms at the tested concentration (Table 2). The venom from crotalid (C. adamanteus) species showed the most potent antibacterial activity and exhibited larger zone of inhibition on B. pseudomallei (FIG. 2) than the venoms of a viperid, D. russelli russelli and an Australian elapid, Pseudechis australis. Crotalid venoms have previously been reported to have broad activity against aerobic gram-positive and negative bacteria (Talan D A, et al, 1991) but the potent antibacterial property of C. adamanteus venom against the drug resistant B. pseudomallei has not been previously reported. The DRR2-PLA₂s enzyme showed very strong antimicrobial action against Staphylococcus aureus, Proteus vulgaris, Enterobacter aerogenes and Proteus mirabilis as shown in FIG. 3. However the DRR2-PLA₂ enzyme showed only a weak antimicrobial effect against Escherichia coli while Pseudomonas aeruginosa lacked any effect at the highest dose tested in the assay systems. The DRR1-PLA₂ exerted only a moderate effect against the tested organisms. However, the DRR2-PLA₂ enzymes proved their potent activity that lead for further studies than the DRR1-PLA₂ enzyme.

The antibacterial effects of crotalid, viperid and elapid venoms established in the present study may likely be due to the cyto-toxins (direct lytic factors) and phospholipase A₂ enzymes contained in those venoms (Baylock R S M, 2000). Association between L-amino acid oxidase activity (LAAO1 and LAAO2) and antibacterial property of Pseudechis australis venoms has previously been suggested (Stiles B G et al, 1991). However, according to our results, the 5 different venoms of C. adamanteus, Daboia russelli russelli, P. australis, P. guttata and A. halys exhibited stronger antimicrobial activity against both strains of B. pseudomallei than that shown by the L-amino acid oxidase enzymes of C. adamanteus and B. atrox venoms, thus suggesting that LAAO activity alone may not be solely responsible for the antibacterial activity of these venoms.

In the present study, we have therefore tested a variety of phospholipase A₂ enzymes purified from different snake venoms against B. pseudomallei (KHW & TES), S. aureus, E. aerogenes, P. vulgaris, P. mirabilis, P. aeruginosa, E. coli, and have found that crotoxin B displays strong antibacterial activity against S. aureus, E. aerogenes, P. aeruginosa, and E. coli and daboiatoxin display the strongest antibacterial activity against both the strains of B. pseudomallei (KHW & TES) (Table 3). Crotoxin B is a basic neurotoxic phospholipase A₂ (Crotalus durissus terrificus) containing three chain protein that enhances the lethal potency of crotoxin (Oliveira D G et al, 2002), while daboiatoxin is a monomeric PLA₂ (Daboia russelli siamensis) with strong neurotoxic and myotoxic activities (Thwin M M et al, 1995). Mulgatoxin (myotoxic PLA₂ of P. australis) and bee (Apis mellifera) venom PLA₂ have also been found to exert significant antibacterial activity against both strains (KHW & TES) of B. pseudomallei.

The total activity and specific activity of phospholipase enzymes from the venom samples and their protein concentrations is shown in Table 4.

Minimum Inhibitory Concentrations (MIC)

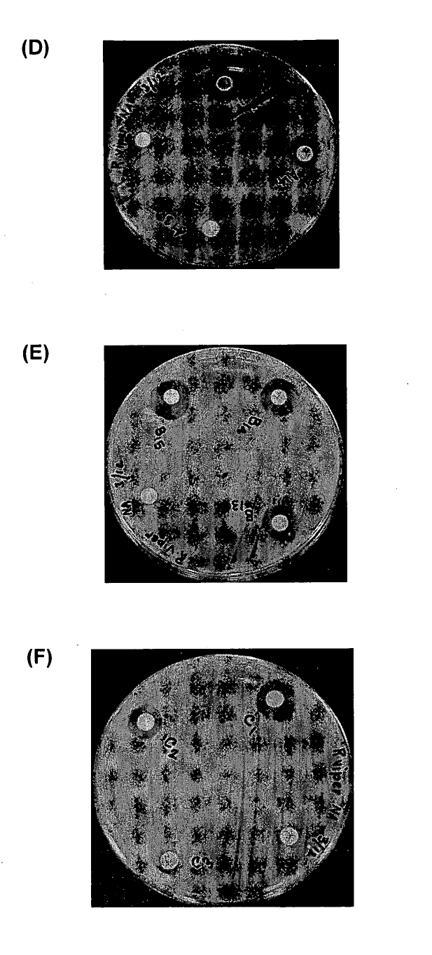

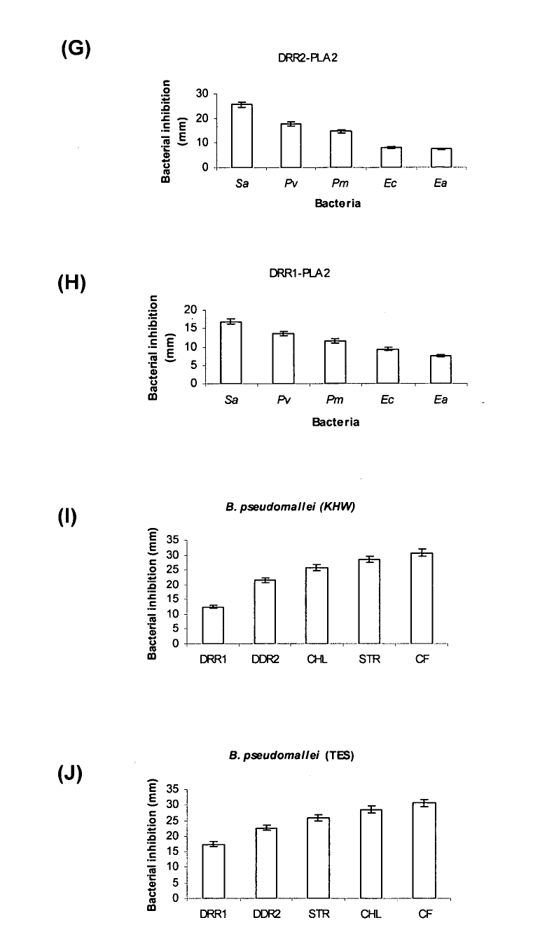

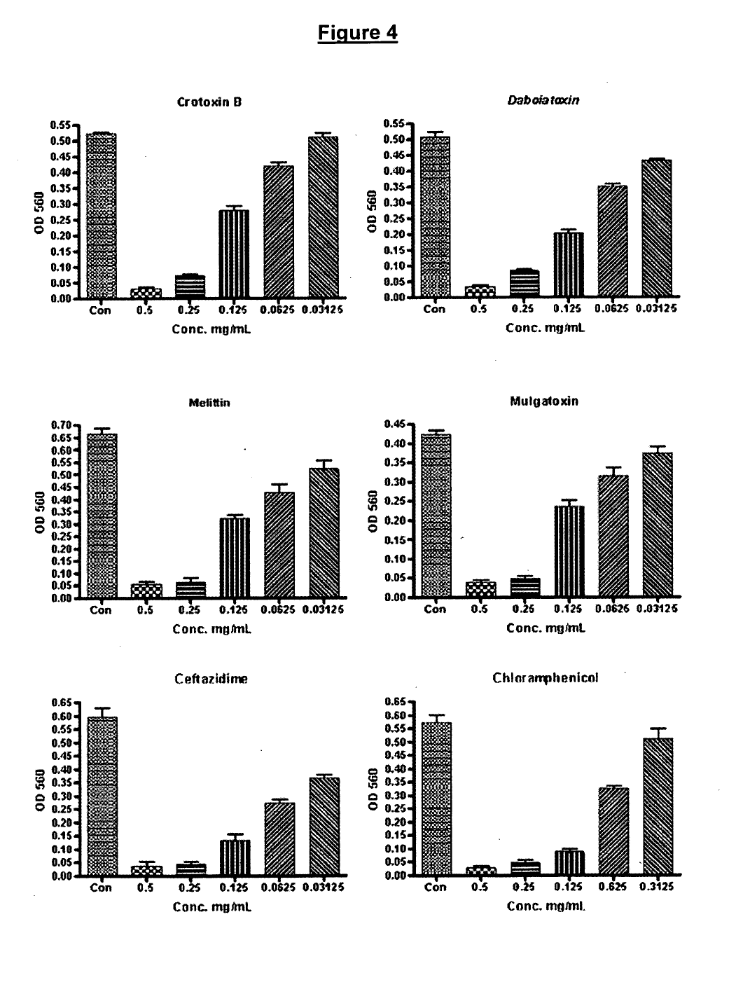

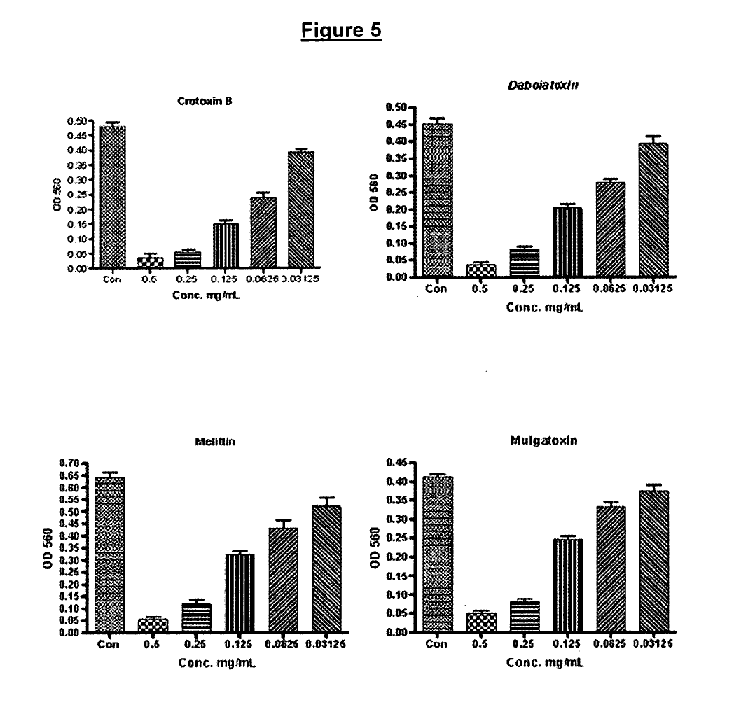

The PLA₂ and DRR2-PLA₂ enzymes showing potent inhibitory activity were further studied for minimum inhibitory concentrations (MICs) (FIGS. 4, 5 and 6). Among those examined, the two purified PLA₂ enzymes (crotoxin B and daboiatoxin) showed stronger inhibitory activity at a lower dilution (MICs 0.25 mg/mL) against B. pseudomallei (TES) than melittin and mulgatoxin. The two isolates (TES and KHW) of B. pseudomallei showed various levels of sensitivity to the two PLA₂ toxins with MICs ranging between 0.5 and 0.03125 mg/ml. The MICs of DRR2-PLA₂ were determined by broth-dilution assay with initial sample concentrations of 42 to 2.56 μM against Gram-positive and Gram-negative bacterial strains including B. pseudomallei (TES & KHW) tested at 0.5-0.03125 μM concentrations. The MIC values are expressed as the lowest concentration that caused 100% bacterial growth inhibition. The DRR2-PLA₂ exhibited marked activity against P. vulgaris, S. aureus, (MICs 5.12 μM) bacteria and P. mirabilis (MICs 10.25 μM), whereas E. coli and E. aerogenes showed weaker inhibitory MICs effect at all dilutions as (42-2.56 μM) shown in FIG. 6A-F. Interestingly, DRR2-PLA₂ exhibited significant inhibition at the lowest dilution (MIC of 5.12 μM) against S. aureus, P. vulgaris and P. mirabilis (MIC of 10.25 μM). The MICs studies have proven the pore-forming action of DRR2-PLA₂ enzyme on bacteria thus confirming the bactericidal activity the enzyme.

The susceptibility of crotoxin B (69%) and daboiatoxin (63%) against TES was more or less equal to that of chloramphenicol and ceftazidime against TES (Table 5). The antibiotics chloramphenicol (70%) and ceftazidime (80%) were highly susceptible against B. pseudomallei TES & KHW, but MIC breaking points of antibiotics were recorded at the lowest dilution (MICs 0.125 mg/mL) than the PLA₂ toxins. Our results corroborate with the previous report on the conventional four-drug regimen which has been replaced by ceftazidime for acute treatment. However, the chlroarmphenicol has also been given for the first 8 weeks of oral treatment (Rajchanuvong A et al, 2000). In the present study, the antibiotic activity was gradually declined at the dilution 0.03125 mg/mL against B. pseudomallei (KHW) and (TES). The rate of resistance of ceftazidime and chloramphenicol against KHW was 20% and 30%, respectively. In a randomised trial previously reported, 161 severe meloidosis patients were treated with ceftazidime (120 mg/kg)+chloramphenicol (100 mg/kg)+doxycycline (4 mg/kg)+TMP/SMX for less than 7 days. The mortality was 37% for ceftazidime and 74% for chloramphenicol (White N J et al, 1989). However, in our study, melittin was slightly more active than mulgatoxin against B. pseudomallei (strain KHW) at MIC 0.25 mg/mL. It showed very weak MICs (FIG. 2, 3) when compared with chloramphenicol and ceftazidime.

Other PLA₂s (ammodytoxin A, Mojave toxin, β-bungarotoxin and taipoxin) however, lacked any activity against both isolates of B. pseudomallei at all tested dilutions (0.5-0.03125 mg/mL). Based on previous studies using cationic antibacterial peptides, cathelicidin-derived peptides had modest MIC against S. maltophilia and A. xylosoxidans (1.0 to >32 mg/mL), but none inhibited Burkholderia cepacia (Bouchier C et al, 1991). Moreover, another cationic peptide (hBD-3) that was proven highly or intermediately effective (MBCs>100 μg/mL) against 23 tested strains did not show any effect against Burkholderia cepacia at 50 μg/mL (Saiman L, et al, 2001). When the MIC of peptide D2A21 was compared with that of the tracheal antimicrobial peptide (TAP), the former peptide displayed greater potency than TAP against P. aeruginosa at 0.125-4 mg/mL, S. aureus at 0.25-4 mg/mL, and Burkholderia cepacia at 32 to >64 mg/mL, respectively (Sahly H, et al, 2003). Relative to the MIC of these cationic antimicrobial peptides, crotoxin B appears to have a much lower dose (MIC 0.25 mg/mL) against B. pseudomallei. MIC value of PLA₂s are more or less equally comparable to that of ceftazidime (MICs 0.125 mg/mL), a drug of choice for melioidosis infections in humans.

TABLE 2
Antibacterial effects of different snake venoms were tested against some clinical isolates of gram-positive
and negative bacteria including Burkholderia pseudomallei (strains 1 & 2) at 100 μg/ml concentration.

| | Snake | Micro-organism (Size of inhibition zone 7 mm in diameter) | | | |
|---|---|---|---|---|---|
| Common name | (Vernacular name) | Sa (+) | Ea (−) | Pv (−) | Pm (−) |
| Elapidae | | | | | |
| Death adder | Acanthophis augtra | 20 ± 0.71 | — | — | — |
| Common death adder | Acanthophis antarcticus * | 21.2 ± 1.93 | — | — | 14.4 ± 0.84 |
| Northern death adder | Acanthophis praelongus | 8.4 ± 0.70 | — | 7.7 ± 0.85 | 8.1 ± 0.44 |
| Desert death adder | Acanthophis pyrrhus | 21.4 ± 1.14 | — | — | 15.5 ± 0.92 |
| Hector | Androctonus australis | — | — | — | — |
| Malayan krait | Bungarus candidus # | 25.1 ± 1.23 | — | — | — |
| Sea snake | Hydrophis cyanocinctus | — | — | — | — |
| Indian cobra | Naja naja naja # | 27.8 ± 1.10 | — | — | — |
| Krefft's tiger snake | Notechis ater ater | — | — | — | — |
| Spitting cobra | Naja sumatrana | 24.4 ± 1.51 | 7.9 ± 0.70 | — | — |
| Cobra | Naja kaouthia | — | — | — | — |
| King brown snake | Pseudechis australis *# | 29.9 ± 0.71 | — | — | — |
| Speckled brown snake | Pseudechis guttata *# | — | 15.2 ± 0.83 | — | 28.8 ± 1.10 |
| Red-bellied black snake | Pseudechis porphyriacus | 22.5 ± 0.50 | — | — | — |
| Collett's snake | Pseudechis colletti | — | 7.2 ± 0.45 | — | — |
| Peninsula brown snake | Pseudonaja inframaggula | 23.3 ± 0.46 | — | — | — |
| Western brown snake | Pseudonaja nuchalis | — | — | — | — |
| Eastern brown snake | Pseudonaja textilis | 15 ± 0.70 | — | — | — |
| Dugite | Pseudechis affinis | — | — | — | — |
| Tiger keelback | Rhabdophis tigrinus | — | — | — | — |
| Viperidae | | | | | |
| Pallas | Agkistrodon halys *# | 24.1 ± 1.23 | 7.4 ± 0.89 | 15.4 ± 0.74 | 17.2 ± 0.83 |
| Diamondback rattlesnake | Crotalus adamanteus *# | 25.4 ± 1.51 | — | 21.7 ± 2.23 | 15.6 ± 0.5 |
| Puff adder | Bitis arietans *# | 26 ± 0.43 | — | — | — |
| West African gaboon viper | Bitis gabonica | 27 ± 0.71 | — | — | — |
| | rhinoceros *# | | | | |
| Russell's viper | Daboia russelli | 29.4 ± 0.89 | 8 ± 0.70 | 26.4 ± 0.98 | 16.8 ± 0.84 |
| | russelli *# | | | | |
| Burmese viper | Daboia russelli | 25.2 ± 0.84 | 14.8 ± 0.83 | — | 7.5 ± 0.50 |
| | siamensis *# | | | | |
| Saw-scaled viper | Echis carinatus *# | 28.6 ± 0.81 | — | — | — |
| The coastal taipan | Oxyuranus scutellatus | — | — | — | — |
| Wagler's pit viper | Trimeresurus wagleri # | 25.2 ± 1.92 | 8.4 ± 0.89 | — | — |
| Apiidae | | | | | |
| Honeybee venom | Apis mellifera | 23.2 ± 1.09 | — | — | — |
| Scorpionidae | | | | | |
| Black scorpion | Androctonus crasicuda | — | — | — | — |
| Scorpion | Buthotus hottentota | — | — | — | — |
| Scorpion | Buthotus hottenota | 15.4 ± 0.89 | — | — | — |
| | hottenota | | | | |
| Chinese red scorpion | Buthus martensii | — | 16.6 ± 0.89 | — | — |
| | Snake | Micro-organism (Size of inhibition zone 7 mm in diameter) | | | |
| Common name | (Vernacular name) | Pa (−) | Ec (−) | Bp (1) | Bp (2) |
| Elapidae | | | | — | — |
| Death adder | Acanthophis augtra | — | — | 16.6 ± 0.43 | 14.3 ± 0.34 |
| Common death adder | Acanthophis antarcticus * | — | — | 8.90 ± 0.23 | 8.47 ± 0.21 |
| Northern death adder | Acanthophis praelongus | 7.2 ± 0.45 | — | 14.7 ± 0.23 | 16.5 ± 0.32 |
| Desert death adder | Acantho phispyrrhus | — | — | — | 8.13 ± 0.14 |
| Hector | Androctonus australis | — | 7.2 ± 0.84 | — | — |
| Malayan krait | Bungarus candidus # | — | — | — | — |
| Sea snake | Hydrophis cyanocinctus | — | — | 12.2 ± 0.16 | 10.2 ± 0.09 |
| Indian cobra | Naja naja naja # | — | — | — | — |
| Krefft's tiger snake | Notechis ater ater | — | — | — | — |
| Spitting cobra | Naja sumatrana | — | 14.4 ± 0.87 | 8.17 ± 0.15 | — |
| Cobra | Naja kaouthia | — | — | 27.7 ± 0.13 | 29.8 ± 0.105 |
| King brown snake | Pseudechis australis *# | — | — | 25.4 ± 0.19 | 26.8 ± 0.109 |
| Speckled brown snake | Pseudechis guttata *# | — | 7.7 ± 0.66 | 16.8 ± 0.15 | 14.2 ± 0.17 |
| Red-bellied black snake | Pseudechis porphyriacus | — | — | — | — |
| Collett's snake | Pseudechis colletti | — | — | 7.7 ± 0.16 | 8.32 ± 0.11 |
| Peninsula brown snake | Pseudonaja inframaggula | — | — | 8.2 ± 0.08 | 9.30 ± 0.11 |
| Western brown snake | Pseudonaja nuchalis | — | — | 14.2 ± 0.09 | 8.10 ± 0.14 |
| Eastern brown snake | Pseudonaja textilis | — | — | — | — |
| Dugite | Pseudechis affinis | — | — | — | — |
| Tiger keelback | Rhabdophis tigrinus | — | — | — | — |
| Viperidae | | | | | |
| Pallas | Agkistrodon halys *# | — | 7.9 ± 0.74 | 20.4 ± 0.14 | 26.4 ± 0.08 |
| Diamondback rattlesnake | Crotalus adamanteus *# | — | — | 18.2 ± 0.16 | 16.2 ± 0.17 |
| Puff adder | Bitis arietans *# | 16 ± 0.83 | — | 16.0 ± 0.19 | 14.5 ± 0.26 |
| West African gaboon viper | Bitis gabonica | — | 7.7 ± 0.85 | 24.4 ± 0.19 | 26.2 ± 0.19 |
| | rhinoceros *# | | | | |
| Russell's viper | Daboia russelli | — | 7.8 ± 0.83 | 8.0 ± 0.14 | 7.5 ± 0.15 |
| | russelli *# | | | | |
| Burmese viper | Daboia russelli | — | — | 29.9 ± 0.12 | 28.6 ± 0.16 |
| | siamensis *# | | | | |
| Saw-scaled viper | Echis carinatus *# | — | — | 16.2 ± 0.15 | 15.6 ± 0.18 |
| The coastal taipan | Oxyuranus scutellatus | — | — | 15.1 ± 0.14 | 16.3 ± 0.14 |
| Wagler's pit viper | Trimeresurus wagleri # | — | — | | |
| Apiidae | | | | | |
| Honeybee venom | Apis mellifera | — | — | 7.22 ± 0.23 | 12.3 ± 0.21 |
| Scorpionidae | | | | | |
| Black scorpion | Androctonus crasicuda | — | — | — | — |
| Scorpion | Buthotus hottentota | — | — | — | — |
| Scorpion | Buthotus hottenota | — | — | — | — |
| | hottenota | | | | |
| Chinese red scorpion | Buthus martensii | — | — | — | — |

| Antibiotics | Concentrations | Sa (+) | Ea (−) | Pv (−) | Pm (−) | Pa (−) | Ec (−) |
|---|---|---|---|---|---|---|---|
| Chloramphenicol (C) | 30 μg/disc | 35.1 ± 1.26 | 27.8 ± 1.09 | 24.6 ± 0.86 | 33 ± 2.34 | 25 ± 0.70 | 31.8 ± 1.64 |
| Streptomycin (S) | 10 μg/disc | 30.3 ± 0.77 | 15.7 ± 0.44 | 18.2 ± 0.83 | 27.8 ± 1.09 | 16.5 ± 0.50 | 29.4 ± 0.89 |
| Penicillin (P) | 10 μg/disc | 17.4 ± 0.89 | 18.3 ± 0.85 | 16.7 ± 0.98 | 27.8 ± 1.10 | 16.6 ± 0.89 | 15.2 ± 0.84 |
| The values are presented as mean ± S.D. (n = 5) represent a venom inhibition zone in mm, including the 7 mm diameter of the disc, after 24 h incubation. The bacterial inoculum per plate contained 3.2 × 10⁸ colony forming units which were spread onto the agar surface with sterile cotton swap. | | | | | | | |
| # Sterile paper discs (7 mm diameter) were placed onto the agar surface and 20 μl of venom (100 μg/ml) added. Micro-organisms: Escherichia coli, Enterobacter aerogenes, Proteus vulgaris, Proteus mirabilis, Pseudomonas aeruginosa, Staphylococcus aureus and Burkholderia pseudomallei (strains 1 & 2); Control (0); No activity (−); | | | | | | | |

* Indicates the broad spectrum of activity;
# Strong activity.

TABLE 3
Antibacterial activity of purified Phospholipase A (PLA₂₎ enzymes from snake venoms

| Phospholipase | Snake | Mol. | Micro-organisms (Size of inhibition zone 7 mm in diameter) | | | |
|---|---|---|---|---|---|---|
| A₂ enzymes | (Vernacular name) | wt. | Conc. | Sa (+) | Ea (−) | Pv (−) |
| L-Amino Acid | Bothrops atrox | — | 0.5 | 27.6 ± 0.73 | — | — |
| Oxidase (LAAO) | | | μmole | | | |
| L- Amino Acid | Crotalus | — | 0.5 | 27.2 ± 0.72 | — | — |
| Oxidase (LAAO) | adamanteus | | μmole | | | |
| Crotoxin A | Crotalus durissus | 23.5 | 0.5 | — | — | — |
| | terrificus | | μmole | | | |
| Crotoxin B | Crotalus durissus | 23.5 | 0.5 | 27.8 ± 1.10 | 21.2 ± 1.93 | — |
| | terrificus *# | | μmole | | | |
| Ammodytoxin A | Vipera ammodytes | 13.8 | 0.5 | — | — | — |
| (ATXc) | ammodytes | | μmole | | | |
| Mojave toxin B | Crotalus scutulatus | 23.5 | 0.5 | — | — | — |
| | scutulatus | | μmole | | | |
| β-Bungarotoxin | Bungarus | 20.5 | 0.5 | — | — | — |
| | multicinctus | | μmole | | | |
| Taipoxin | Oxyuranus scutellatus | 45.6 | 0.5 | — | — | — |
| | scutellatus | | μmole | | | |
| Mulga toxin | Pseudechis | 13.2 | 0.5 | — | 8.4 ± 0.89 | — |
| | australis # | | μmole | | | |
| Daboiatoxin (DaTx) | Daboia russelli | 13.6 | 0.5 | 14.3 ± 0.84 | — | — |
| | siamensis # | | μmole | | | |
| Bee venom PLA₂ | Apis mellifera # | — | 0.5 | 13.4 ± 0.83 | — | — |
| | | | μmole | | | |

| Phospholipase | Snake | Micro-organisms (Size of inhibition zone 7 mm in diameter) | | | | |
|---|---|---|---|---|---|---|
| A₂ enzymes | (Vernacular name) | Pm (−) | Pa (−) | Ec (−) | Bp (1) | Bp (2) |
| L-Amino Acid | Bothrops atrox | 26.9 ± 0.60 | — | — | — | — |
| Oxidase (LAAO) | | | | | | |
| L- Amino Acid | Crotalus | 24.9 ± 1.19 | — | — | — | — |
| Oxidase (LAAO) | adamanteus | | | | | |
| Crotoxin A | Crotalus durissus | — | — | — | — | — |
| | terrificus | | | | | |
| Crotoxin B | Crotalus durissus | — | 24.6 ± 0.86 | 25 ± 0.70 | 24.8 ± 0.089 | 27.6 ± 0.133 |
| | terrificus *# | | | | | |
| Ammodytoxin A | Vipera ammodytes | — | — | — | — | — |
| (ATXc) | ammodytes | | | | | |
| Mojave toxin B | Crotalus scutulatus | — | — | — | — | — |
| | scutulatus | | | | | |
| β-Bungarotoxin | Bungarus | — | — | — | — | — |
| | multicinctus | | | | | |
| Taipoxin | Oxyuranus scutellatus | — | — | — | — | — |
| | scutellatus | | | | | |
| Mulga toxin | Pseudechis | — | — | — | 20.5 ± 0.075 | 22.7 ± 0.117 |
| | australis # | | | | | |
| Daboiatoxin (DaTx) | Daboia russelli | — | — | — | 24.8 ± 0.103 | 26.2 ± 0.121 |
| | siamensis # | | | | | |
| Bee venom PLA₂ | Apis mellifera # | — | — | — | 18.3 ± 0.089 | 20.4 ± 0.075 |
| The values are presented as mean ± S.D. (n = 5) represent a PLA₂s inhibition zone in mm diameter of the disc, after 24 h incubation. The bacterial inoculum per plate contained 3.2 × 10⁸ cfu/ml forming units which were spread onto the TS agar surface with sterile cotton swap. | | | | | | |
| # Sterile paper discs (7 mm diameter) were placed onto the TS agar surface and 20 μl of enzymes (0.5 μM concentration) added, Control (0); No activity (−). Micro-organisms: Escherichia coli, Enterobacter aerogenes, Proteus vulgaris, Proteus mirabilis, Pseudomonas aeruginosa, Staphylococcus aureus and Burkholderia pseudomallei (strains 1 & 2); Control (0); No activity (−); | | | | | | |

* Indicates the broad spectrum of activity;
# Strong activity.

TABLE 4
Total and specific activity of phospholipase enzyme (PLA₂)
activity and protein contents of different venom samples.

| | PhospholipaseA₂ activity | | | |
|---|---|---|---|---|
| | Total | Specific | Protein concentration | |
| | activity | activity | Protein | Yield of |
| | (μmoles/ | (μmoles/ | (Net | protein |
| Species | min) | min/mg) | OD 595 nm) | (mg/ml) |
| Elapidae | | | | |
| Acanthophis | 3.74 | 74.5 ± 0.5 | 0.104 | 0.1 |
| augtra | | | | |
| Acanthophis | 190 | 487.5 ± 0.96 | 0.593 | 0.78 |
| antarcticus | | | | |
| Acanthophis | 241.2 | 1416 ± 3.84 | 0.267 | 0.34 |
| praelongus | | | | |
| Acanthophis | 138 | 1150 ± 0.98 | 0.207 | 0.24 |
| pyrrhus | | | | |
| Androctonus | 22.2 | 85.2 ± 0.27 | 0.389 | 0.52 |
| australis | | | | |
| Bungarus | 34.9 | 166.3 ± 0.56 | 0.282 | 0.42 |
| candidus | | | | |
| Hydrophis | 3.3 | 40.5 ± 0.56 | 0.133 | 0.16 |
| cyanocinctus | | | | |
| Naja naja | 293.4 | 1333 ± 1.7 | 0.337 | 0.44 |
| naja | | | | |
| Notechis | 41.96 | 183.1 ± 0.59 | 0.346 | 0.46 |
| aterater | | | | |
| Naja | 406.5 | 903.5 ± 0.6 | 0.833 | 0.90 |
| sumatrana | | | | |
| Naja | 228.4 | 1904 ± 3.8 | 0.165 | 0.24 |
| kaouthia | | | | |
| Pseudechis | 434.5 | 3949 ± 3.2 | 0.162 | 0.22 |
| australis | | | | |
| Pseudechis | 308.5 | 791 ± 2.0 | 0.592 | 0.78 |
| guttata | | | | |
| Pseudechis | 726.7 | 3303 ± 1.7 | 0.372 | 0.44 |
| porphyriacus | | | | |
| Pseudechis | 15.7 | 111.8 ± 0.89 | 0.148 | 0.28 |
| colletti | | | | |
| Pseudonaja | 1995 | 5945 ± 26.6 | 0.546 | 0.66 |
| inframaggula | | | | |
| Pseudonaja | 162.8 | 361.8 ± 0.6 | 0.768 | 0.90 |
| nuchalis | | | | |
| Pseudonaja | 416.8 | 832.3 ± 0.6 | 0.83 | 1 |
| textilis | | | | |
| Pseudechis | 218.7 | 376.8 ± 0.9 | 0.937 | 1.16 |
| affinis | | | | |
| Rhabdophis | 36.3 | 259.1 ± 0.42 | 0.186 | 0.28 |
| tigrinus | | | | |
| Oxyuranus | 1275.7 | 6075 ± 0.98 | 0.316 | 0.63 |
| scutellatus | | | | |
| Viperidae | | | | |
| Agkistrodon | 86.5 | 157.4 ± 0.20 | 0.841 | 1.1 |
| halys | | | | |
| Bitis | 124.1 | 248.2 ± 0.27 | 0.402 | 0.54 |
| arietans | | | | |
| Bitis | 126.5 | 452.4 ± 0.57 | 0.46 | 0.56 |
| gabonica | | | | |
| rhinoceros | | | | |
| Bothrops | 3.8 | 6.36 ± 0.06 | 0.271 | 1.2 |
| atrox | | | | |
| (L-amino | | | | |
| acid oxidase) | | | | |
| Crotalus | 34.2 | 201.3 ± 0.21 | 1.211 | 0.34 |
| adamanteus | | | | |
| (L-amino | | | | |
| acid oxidase) | | | | |
| Crotalus | 236.4 | 619.4 ± 0.46 | 1.355 | 1.56 |
| adamanteus | | | | |
| Echis | 53.4 | 106.5 ± 0.49 | 1.19 | 1.4 |
| carinatus | | | | |
| Daboia | 392.8 | 785.2 ± 0.40 | 1.27 | 1.36 |
| russelli | | | | |
| russelli | | | | |
| Daboia | 262.4 | 524.4 ± 0.44 | 1.104 | 1.24 |
| russelli | | | | |
| siamensis | | | | |
| Trimeresurus | 4.6 | 38.2 ± 0.26 | 0.204 | 0.24 |
| wagleri | | | | |
| Apiidae | | | | |
| Apis | 3.7 | 20.5 ± 0.1 | 0.283 | 0.36 |
| mellifera | | | | |
| Scorpionidae | | | | |
| Androctonus | 3.6 | 69.5 ± 0.3 | 0.075 | 0.104 |
| crasicuda | | | | |
| Buthotus | 4.4 | 10.4 ± 0.2 | 0.674 | 0.86 |
| hottenlota | | | | |
| Buthotus | 5.1 | 38.8 ± 0.1 | 0.178 | 0.26 |
| hottenota | | | | |
| hottenota | | | | |
| Buthus | 4.6 | 90.4 ± 0.2 | 0.068 | 0.102 |
| martensii | | | | |

Total activity of PLA₂ enzyme estimated from the whole venoms (μmoles/min).
Phospholipase A₂ enzymatic activity (μmoles/min/mg).
Values are presented as mean ± S.D. (n = 10) of ten replicates.

TABLE 5
MIC breakpoints for ceftazidime and chloramphenicol when compared to that of purified PLA₂s enzymes.

| Phospholipase | MIC | | | | | | MIC | | | | | |
|---|---|---|---|---|---|---|---|---|---|---|---|---|
| A₂ enzymes | mg/mL | B. pseudomallei (strain KHW) | | | | | mg/mL | B. pseudomallei (strain TES) | | | | |
| (PLA₂s) | Ctrl | 0.5 | 0.25 | 0.125 | 0.0625 | 0.03125 | Ctrl | 0.5 | 0.25* | 0.125^{a} | 0.0625 | 0.03125 |
| Crotoxin B (CB) | 0.65 | 0.012 | 0.04 | 0.33 | 0.43 | 0.56 | 0.73 | 0.05 | 0.06 | 0.33 | 0.43 | 0.52 |
| | | (64%) | (61%) | (32%) | (32%) | (9%) | | (73%) | (69%) | (40%) | (30%) | (21%) |
| Daboiatoxin | 0.65 | 0.26 | 0.09 | 0.37 | 0.49 | 0.55 | 0.68 | 0.03 | 0.05 | 0.24 | 0.36 | 0.43 |
| (DbTx) | | (39%) | (56%) | (28%) | (16%) | (10%) | | (65%) | (63%) | (20%) | (8%) | (1%) |
| Bee venom PLA₂ | 0.48 | 0.038 | 0.056 | 0.15 | 0.24 | 0.39 | 0.44 | 0.03 | 0.06 | 0.27 | 0.36 | 0.41 |
| | | (38%) | (42%) | (33%) | (24%) | (9%) | | (45%) | (38%) | (17%) | (8%) | (3%) |
| Mulgatoxin | 0.46 | 0.07 | 0.083 | 0.21 | 0.28 | 0.39 | 0.43 | 0.04 | 0.07 | 0.26 | 0.32 | 0.37 |
| | | (39%) | (37%) | (25%) | (18%) | (7%) | | (40%) | (37%) | (18%) | (12%) | (7%) |
| Chloramphenicol | 0.67 | 0.01 | 0.03 | 0.07 | 0.42 | 0.51 | 0.77 | 0.026 | 0.04 | 0.07 | 0.34 | 0.37 |
| | | (68%) | (64%) | (60%) | (10%) | (1%) | | (74%) | (73%) | (70%) | (41%) | (38%) |
| Ceftazidime | 0.76 | 0.08 | 0.05 | 0.04 | 0.24 | 0.44 | 0.89 | 0.01 | 0.03 | 0.09 | 0.35 | 0.49 |
| | | (58%) | (61%) | (62%) | (24%) | (22%) | | (88%) | (86%) | (80%) | (47%) | (33%) |

*MIC values are given as mean of five replicates (n = 5), the bacterial inoculums per plate contained 3.2 × 10⁸ cfu/mL forming units/well, Control (bacterial inoculums);
^{a}The ceftazidime breaking points (MICs 0.125 mg/mL) against TES,
*The PLA₂ toxin breaking points (MICs 0.250 mg/mL) against both the strains of B. pseudomallei after 24 h incubation.

Cytotoxicity (XTT Based Assay) for Dose Optimization

When PLA₂ activity was examined, the highest activity was found in the Australian elapid venoms (Oxyuranus scutellatus, Pseudonaja inframaggula) followed by Pseudechis australis, Pseudechis porphyriacus, Naja kaouthia, Naja naja naja, Acanthophis praelongus and Acanthophis pyrrhus respectively. In contrast, the remaining venoms of the Apiidae and Scorpionidae showed relatively less phospholipase A₂ activity than the viperidae venoms.

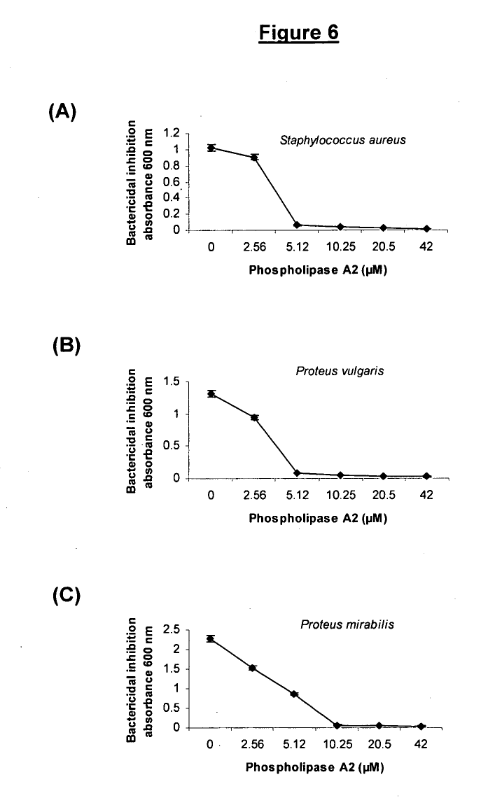

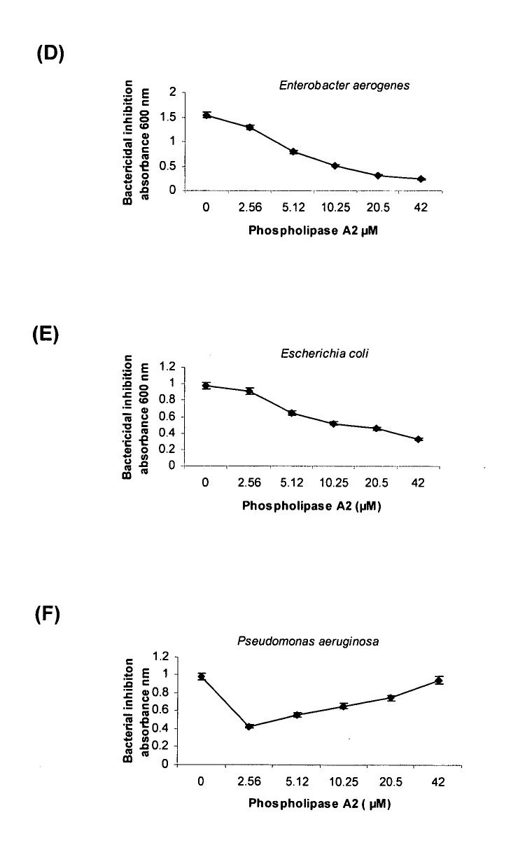

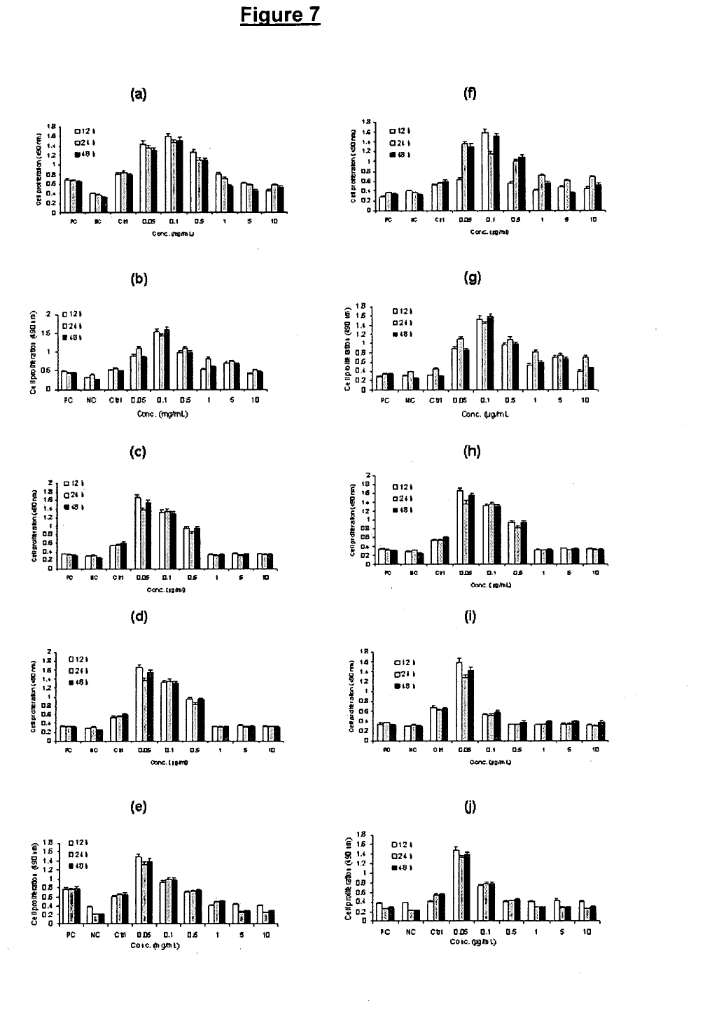

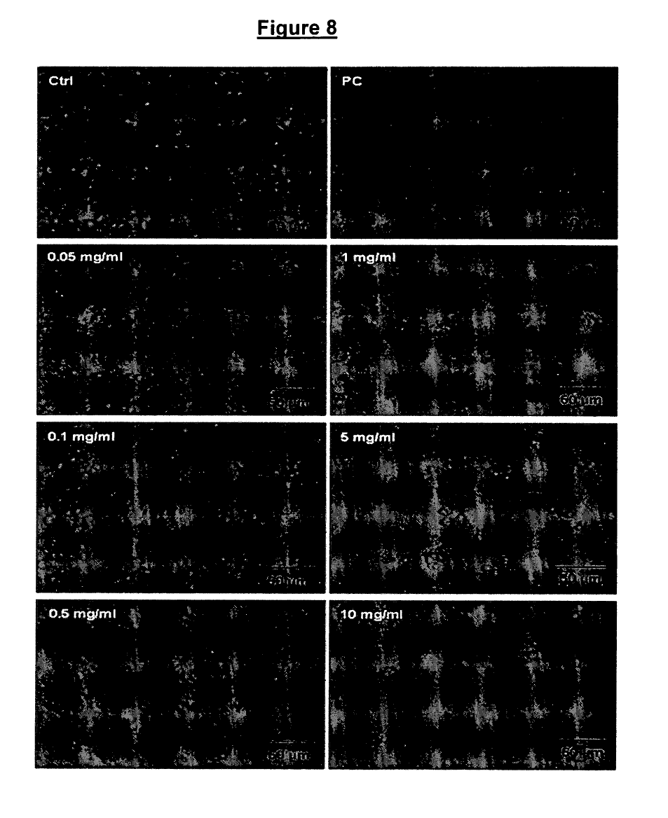

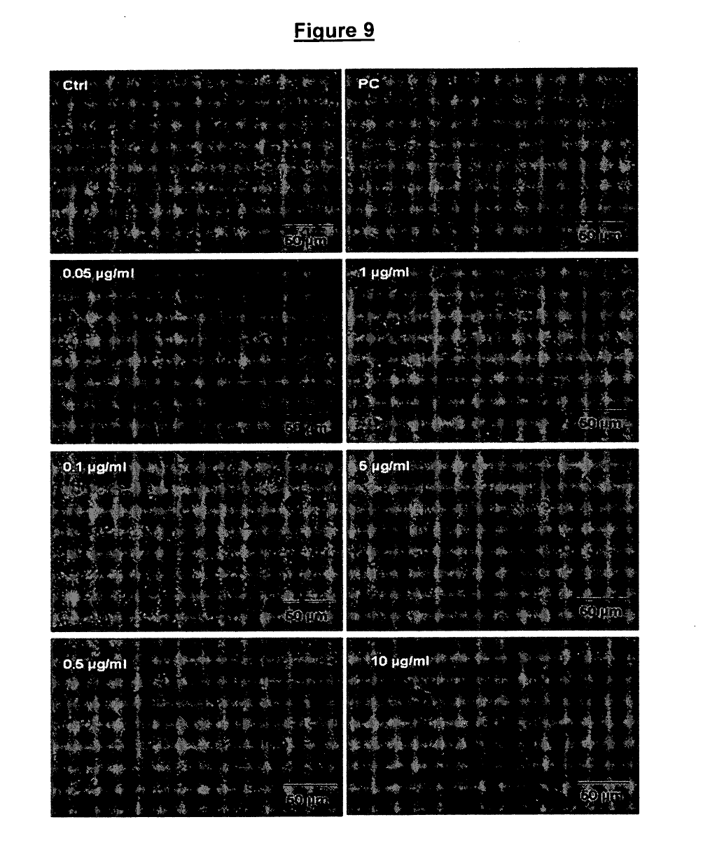

The survival bar resulting from the XTT assay shows that all five venoms (C. adamanteus, B. gabonica, P. australis, D. russelli russelli and A. halys) do not have cytotoxic effects on the proliferation of cells up to 0.5 mg/mL concentration (FIG. 7a-e). However, the higher concentrations (1, 5 and 10 mg/mL) of the five venoms (C. adamanteus, B. gabonica, P. australis, D. russelli russelli and A. halys) showed severe morphological changes of the cell lines such as membrane disruption and significant cell lyses when compared with control and the positive control (FIG. 8). In contrast, the purified PLA₂s, crotoxin B and daboiatoxin, did not change the viability (FIG. 7f-g) of cells up to 0.05-10 μg/mL concentrations as compared to the control. The cell viability and morphology of macrophage cells were shown (FIG. 9) after exposure to crotoxin B in a dose- and time-dependent manner.

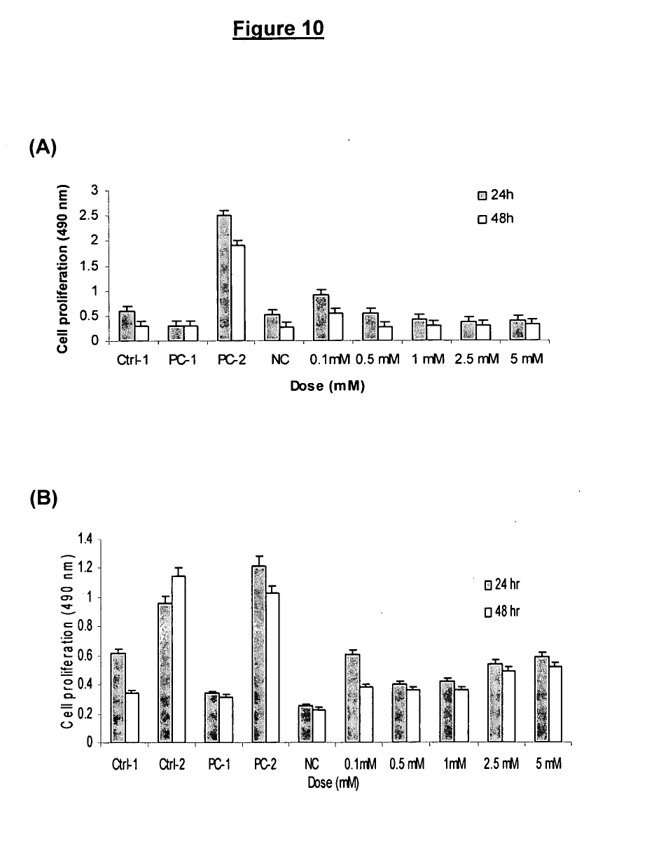

The XTT assay results further showed that THP-1 cell survival decreases with increasing concentrations of DRR2-PLA₂ with EC₅₀ calculated as ˜1 mM concentrations. DRR2-PLA₂ did not affect the cell viability at 1 mM concentrations (FIGS. 10A&B).

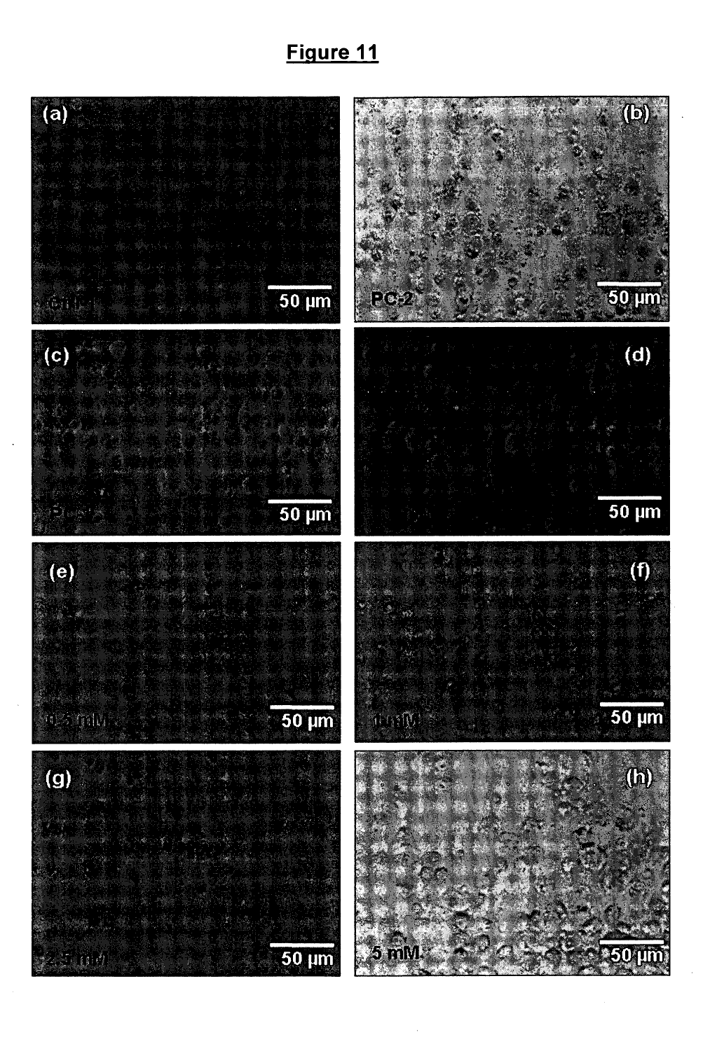

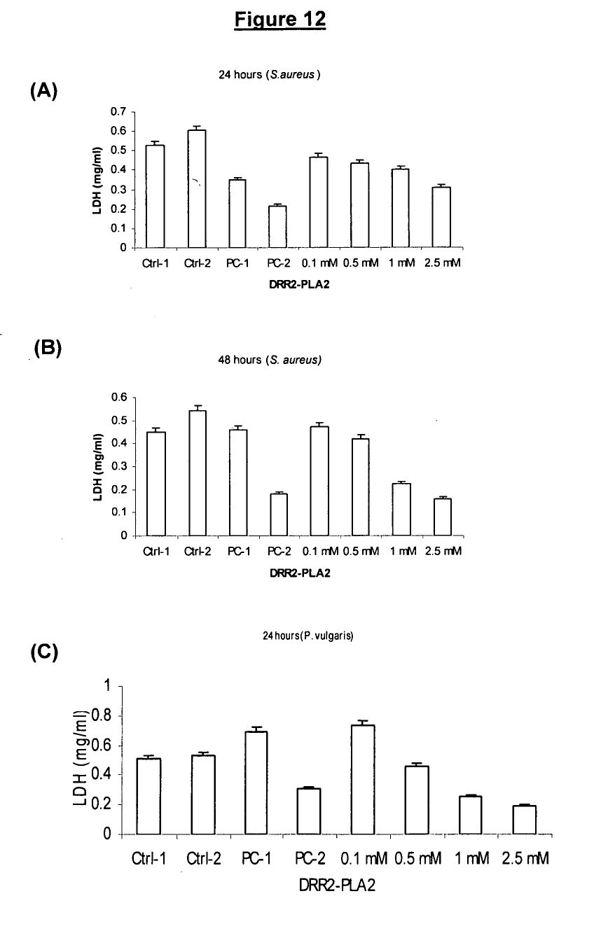

The incubation of THP-1 cells with DRR2-PLA₂ did not affect cell viability up to 1 mM concentrations (FIGS. 11a-h). Morphological changes of the cells indicate that the cells remain intact with moderate swelling but without any membrane disruption or cell lysis at 2.5 mM (FIG. 11g). The cell proliferation decreases with increasing concentrations of DRR2-PLA₂ with the EC₅₀ calculated as ˜2.5 mM, the morphological changes of the cells remain intact, prominent, and lysis. However, the growth of THP-1 cells was not affected especially at the optimal dose of DRR2-PLA₂ (0.1 mM), as shown in FIG. 11d. Cell death is evident at higher concentrations (5 mM) of DRR2-PLA₂ in a time dependent assay (FIG. 11h). The growth of monocytic cells was not affected particularly at the optimal dose of DRR2-PLA₂ treated with THP-1 cells. The positive control 90% of the cell death was occurred after the treatment with 10% trition ×100 used as a positive control then the normal control cells. However, the growth of THP-1 cells was not affected especially up to 1 mM concentration of DRR2-PLA₂ Therefore, the DRR2-PLA₂ was selected at this optimum dose 0.1 mM (as recorded by XTT assay) to study the differential expression of genes in the monocytes of THP-1 cells (Human macrophage).

LDH Assay

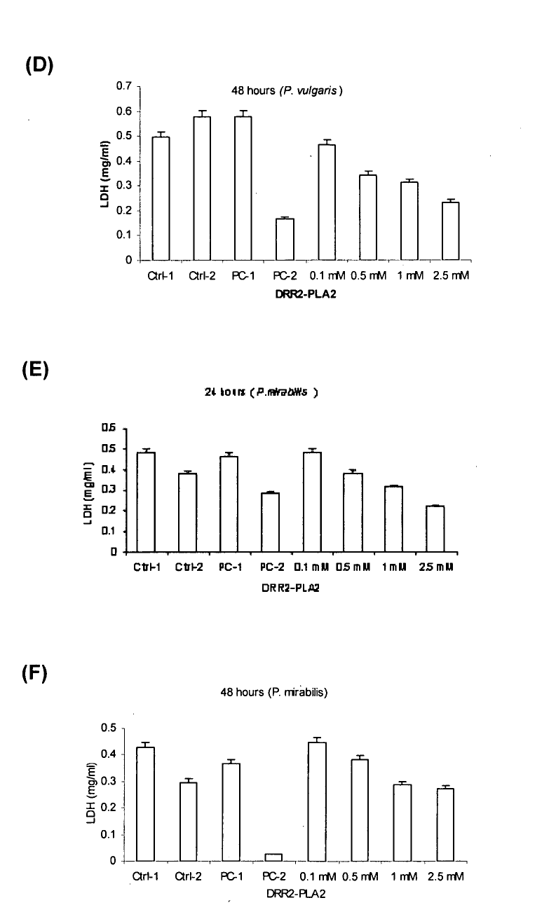

The quantification of cell death and cell lysis, based on the measurement of lactate dehydrogenase (LDH) activity released from the cytosol of damaged cells into the supernatant. The assay was performed in triplicate (n=3). The invasion cytotoxicity (LDH) results revealed that the infected cells (THP-1) exposed to DRR2-PLA₂ showed no cytotoxicity up to 1 mM concentration (FIG. 12) against S. aureus, P. vulgaris and P. mirabilis. Significant cell death is evident at higher concentration (2.5 mM), of DRR2-PLA₂ in time and dose dependent manner (24 & 48 h) as a result more LDH release into the media. However, the growth of THP-1 cells was not affected especially at the optimal dose of DRR2-PLA₂ (1 mM). The optimum dose that inhibited bacterial proliferation after the treatment with DRR2-PLA₂ enzymes did not affect THP-1 cells.

Hemolytic Assay

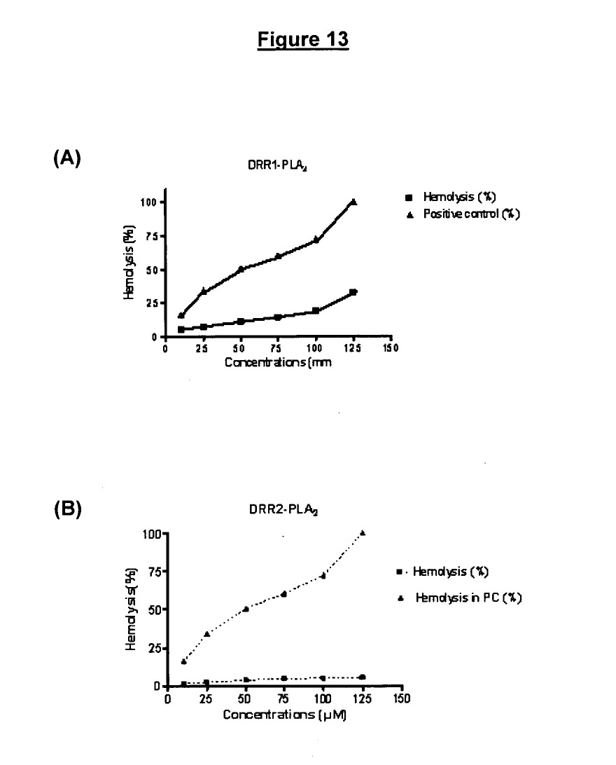

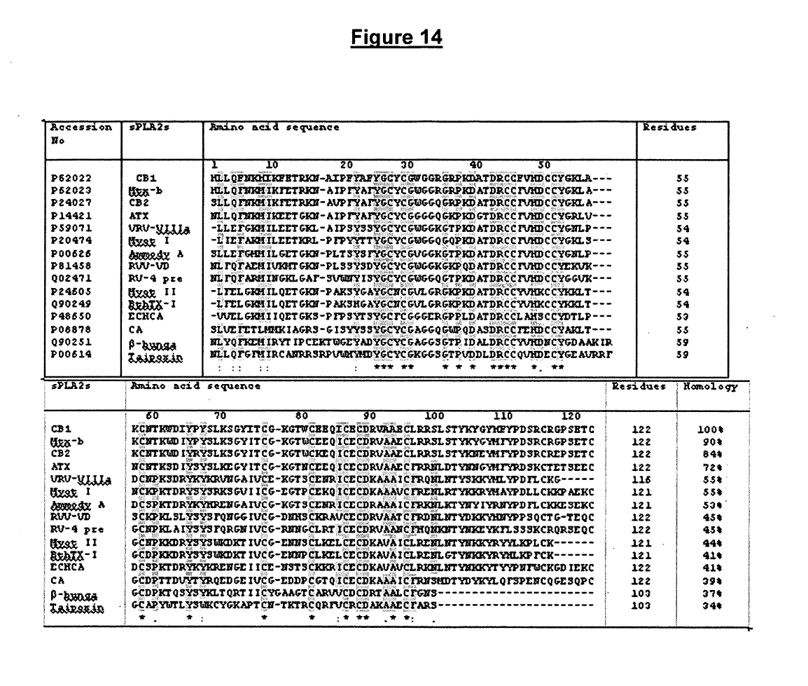

The DRR2-PLA₂ was incubated with human erythrocytes of normal volunteers at the different concentrations of enzyme (10-125 μM) and hemolysis was measured. DRR2-PLA₂ did not exhibit hemolytic activity on human erythrocytes up to a concentration of 125 μM (FIG. 13). However, 100% significant hemolytic activity was observed with 10% Triton X-100 used as a positive control compared with the normal control.

N-Terminal Sequencing

DRR1-PLA₂ and DRR2-PLA₂ was reduced and pyridylethylated prior to sequence analysis. The N-terminal 37 amino acid sequences of DRR2-PLA₂ were determined and shown the multiple (Table 1a & b) alignment with several selected other RV-PLA₂s from Russell's viper. The sequence comparison shows that DRR2-PLA₂ shares greatest sequence identity (70 residues, 80%) with a PLA₂ from the viper groups, and a high degree of sequence homology with the group RV-VIIIA PLA₂s, particularly 40 residues of N-terminal amino acid sequence of DRR2-PLA₂ did not match with existing PLA₂ due the post translation modification and also the Asp-49 enzymes from several hydrophobic enzymes, are apparent.

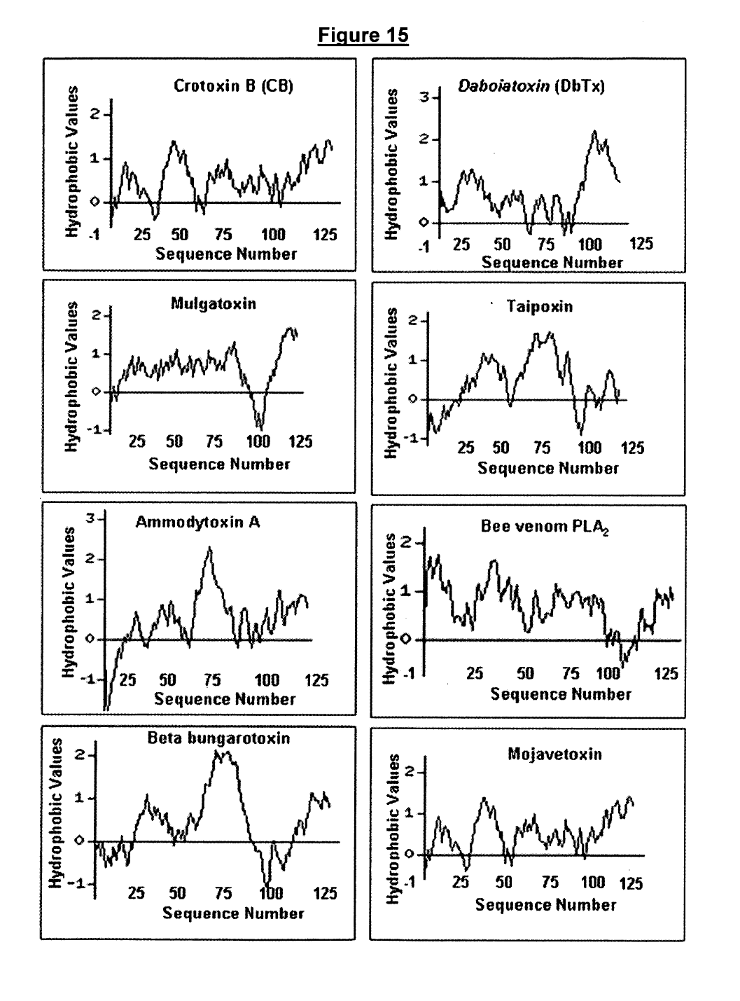

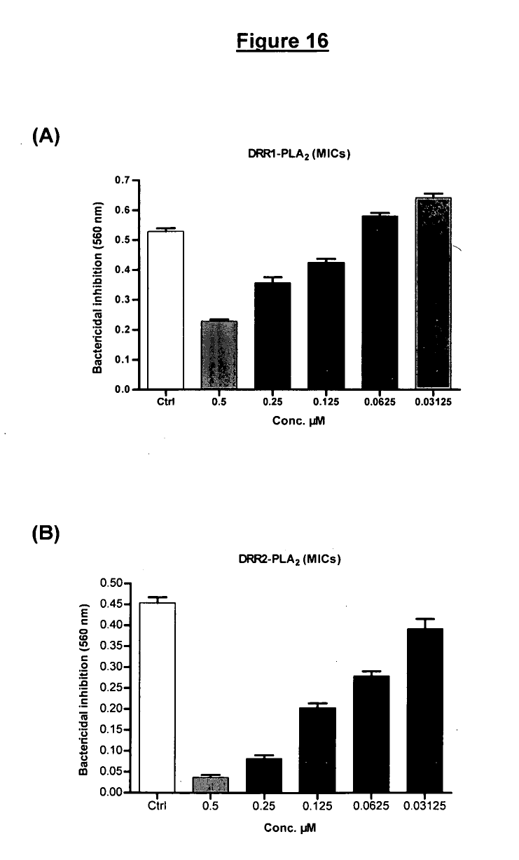

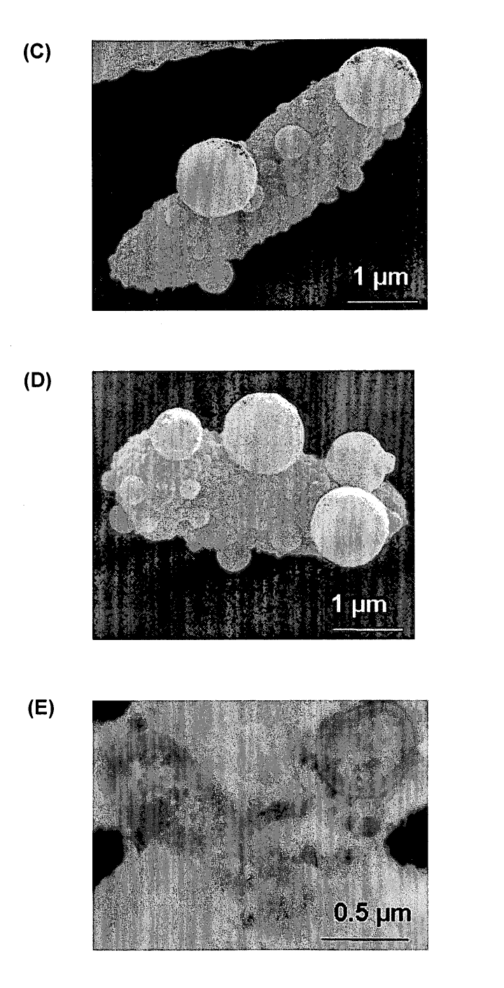

A comparison of the N-terminal sequences of crotoxin B with other snake venom PLA₂s shows that most amino acid residues are conserved in Ca⁺² binding and catalytic network regions (FIG. 14). The C-terminal segment of crotoxin B, on the other hand, shows a modest difference in the amino acid residues as compared with the C-terminal sequences of other venom PLA₂s. Moreover, comparison of the hydropathic profiles of different snake venom PLA₂s reveals that the C-terminal segment of crotoxin B is relatively more hydrophobic than those of other PLA₂s (FIG. 15). This cationic hydrophobic nature of crotoxin B phospholipase A₂ enzyme may most likely be responsible for the strong antimicrobial action seen on B. pseudomallei.

The present study indicates that the purified crotoxin B, daboiatoxin and DDR2-PLA₂ enzymes possess strong antibacterial activity against wide range of potent Gram-positive and Gram-negative bacteria. Thus, the studies provide new insights into the ultra-structural features of novel membrane damaging and pore formation induced by DRR2-PLA₂ (Indian, Russell's viper) and also these molecules neither haemolytic action on human erythrocytes nor cytotoxic on monocytic cells (THP-1). The present studies proved the non-cytotoxic forms of DRR2-PLA₂, as a new novel enzyme has potent microbicidal activities on variety of Gram (+ and −) bacteria.

The fact that viperidae (Crotalus adamanteus, Daboia russelli russelli) and elapidae (Pseudechis australis) venoms display more potency than other venoms may be due to the PLA₂ enzymes present.

Microarray Analysis

In vitro antiburkholderial activity: Antibacterial susceptibility of Daboia russelli russelli-2 phospholipase A₂ (DRR2-PLA₂) enzyme was assayed against B. speudomallei (strains TES & KHW) and their activity compared within the multi-drug resistant (MDR) strains. The DRR2-PLA₂ has more active against KHW (B. speudomallei) than TES strains. The inhibitory potential of DRR2-PLA₂ showed as equal to that of standard drugs streptomycin, chloramphenicoil and ceftazidime. The inhibitory potential of DRR1-PLA₂ and DRR2-PLA₂ was further quantified by TS broth dilution method (0.5-0.3135 μM) as shown in FIG. 16(A)-(B). The MICs result was revealed that the DRR2-PLA₂ exerted most significant inhibition against KHW strains of Burkholderi pseudomallei at the lowest dilutions (MICs 0.125 μM) when compared that of control. The DRR1-PLA₂ was found only weak inhibitory (MICs) effect against multi-drug resistant strains of B. pseudomallei (FIG. 16(A)) at all tested concentrations. The mechanism of antibacterial effects proved by ultra-structural studies, scanning electron microscopic (SEM) picture of Burkholderia pseudomallei after the treatment with DRR2-PLA₂ was induced pore formation on clinical isolates of MDR KHW strain of B. pseudomallei (FIG. 16(C), FIG. 16(D)). The TEM microscopic pictures were also showed the clear evidence of pore formation on B. pseudomallei (KHW) bacteria after the treatment of DRR2-PLA₂ when compared to control (FIG. 16(E) and FIG. 16(F)). The bacilli showed smooth morphology and clear visible cell wall of B. pseudomallei bacteria after 24 hours incubation in normal control (NC). Transmission electron microscopic picture showed the bacterial membrane was disintegrated by the DRR-PLA₂ toxin after the treatment. The ultra-structural (SEM) studies have proved that the DRR2-PLA₂ as a pore-forming properties. Similarly the TEM results were also revealed that the cellular changes of cell lysis, membrane disintegration and pore formation in the present findings.

Gene Expression

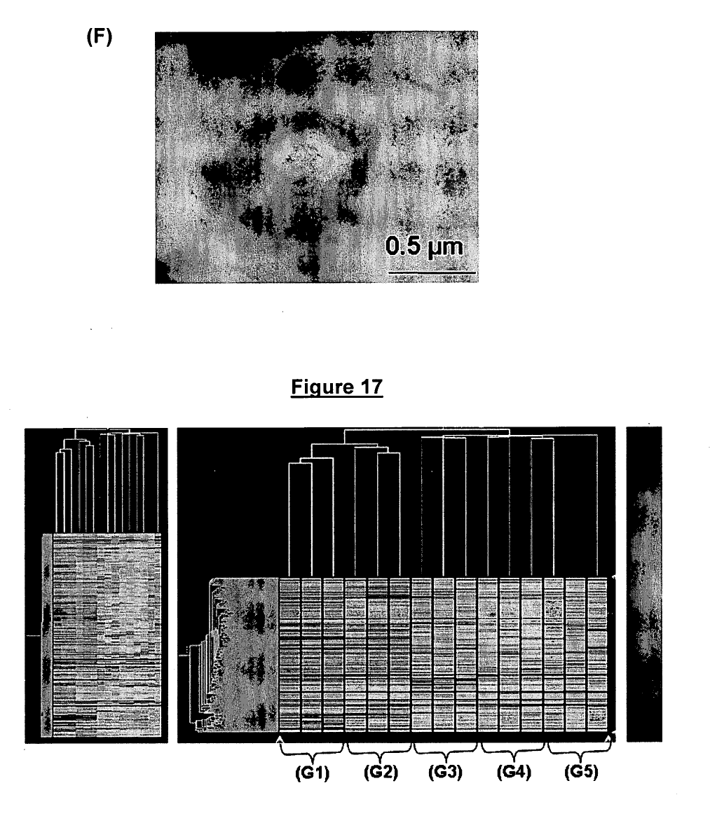

The cluster analysis (FIG. 17) shows the overall expression pattern and biological correlation of replicates (n=3 chips of transcript used per treatment) in cellular response to DRR2-PLA₂ treatment only one time point (24 h). FIG. 17 showed that the greatest number of up-regulated genes (114 out of 2912) were secreted proteins, including cytokines (COX) and tumor necrosis factor alpha (TNF-alpha) known to be induced by bacterial stimulation of macrophages. The strongest up-regulation was recorded for interlukin-12 (IL-12), interferon gamma (IFγ) and chemokines compared to controls respectively. The extensive amount of data accumulated from the GeneChip study and the detail analysis will show us the exact mechanisms of action of this peptide.

The global view of differential gene expression, focused on genes were either consistently increased or decreased in all groups (G1-G5) of experiment, for the grouping details see the Table 6.

| | | Experimental | |
|---|---|---|---|
| S. No | Groups | design | Treatment |
| 1 | Group (I) | Control | Cells only (THP-1) |
| 2 | Group (II) | Peptide control | Cells + DRR2-PLA2 |
| 3 | Group (III) | Disease control | Cells + B. pseudomallei |
| 4 | Group (IV) | Treatment | Cells + B. pseudomallei + |
| | | | DRR2-PLA2 |
| 5 | Group (V) | Drug treatment | Cells + B. pseudomallei + |
| | | | Ceftazidime |

GeneSpring software was used to analyze data, the gene expression changed by 2.5 fold in at least tow pair wise comparisons were taken as significant. The highly and tightly clustered patterns were identified the total genes expression changes after the DRR2-PLA₂ treatment followed by a dramatic increase or decrease. In total 2919 genes (P<0.05) were analyses for fold changes for up and down regulation as induced by DRR2-PLA₂. Using a cutoff value of ≧±2.5 fold change in transcript abundance in all the experiments, a total of 114 genes were selected as relative responsive to DRR2-PLA₂. DRR2-PLA₂ responsive genes were divided into functional classes based on the gene ontology including biological function, cellular component and molecular function in all groups.

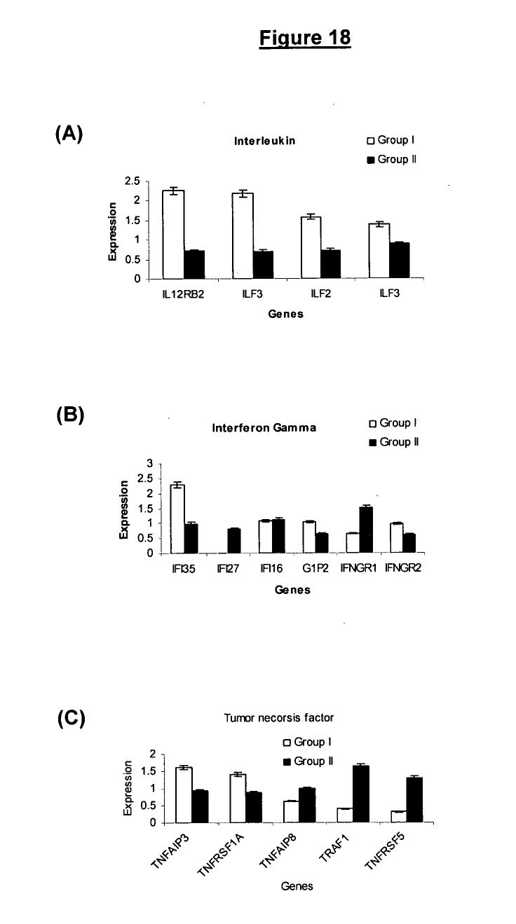

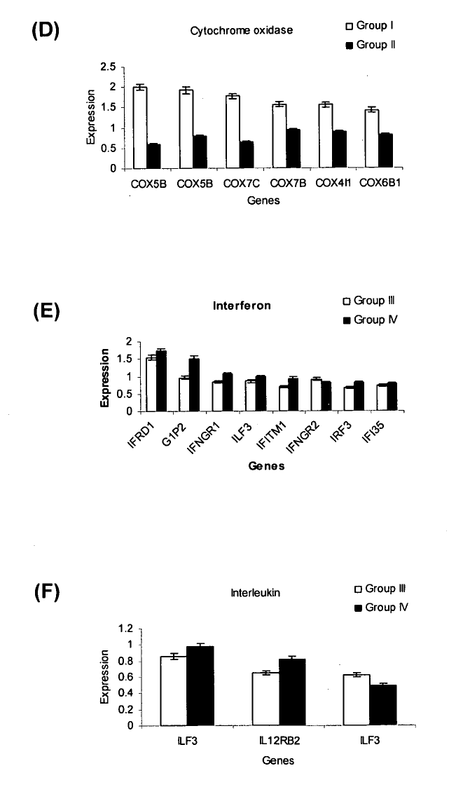

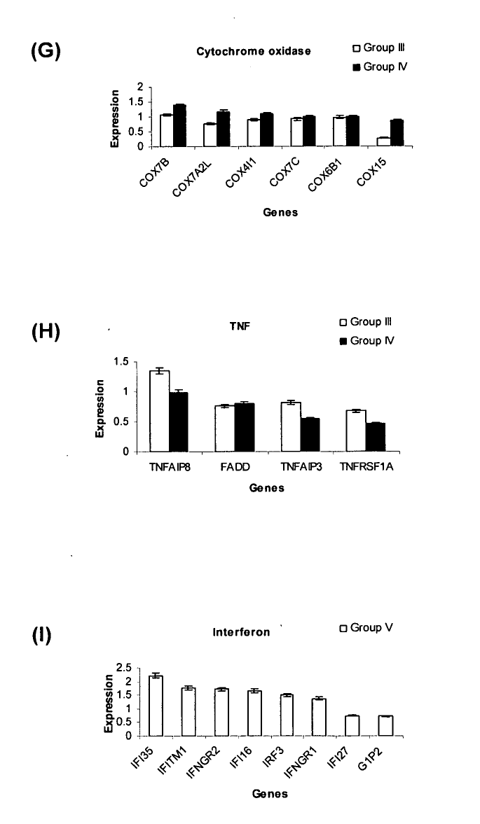

The selected genes were compared within the groups (G1-G2), (G3-G4), (G5) (FIG. 18). The inflammatory related genes like tumor necrosis factors (TNF), cytochrome oxidases (COX), non-inflammatory genes interferon gammas (IF) and interleukins (IL) were strongest up-regulated in the G1 used as a control (THP-1 cells only). The DRR2-PLA₂ individually treated G2, all the inflammatory genes (TNF &, COX) were down regulated than the IL & IF genes (cell+treated with DRR2-PLA₂). Interestingly, the all inflammation induced genes were significantly down regulated in the infected cells treated with DRR2-PLA₂ (THP-1+B. pseudomallei+DRR2-PLA₂) and interferon gammas (IF) and interleukins (IL) genes were highly up-regulated and the genes involved for immunity against infection of bacteria (G4), whereas in the diseases control (G3), TNF &, COX genes were up regulated that induced inflammation during the infection of bacteria (B. pseudomallei). In G5 majority of the genes were up regulated in the cells treated with standard drugs (THP-1+B. pseudomallei+Ceftazidime) than the inflammatory (TNF &, COX) genes, the drug used as a treatment choice for the human infection of B. pseudomallei. Genes were altered in the drug as well as peptide treated.

Electron Microscopic Analysis

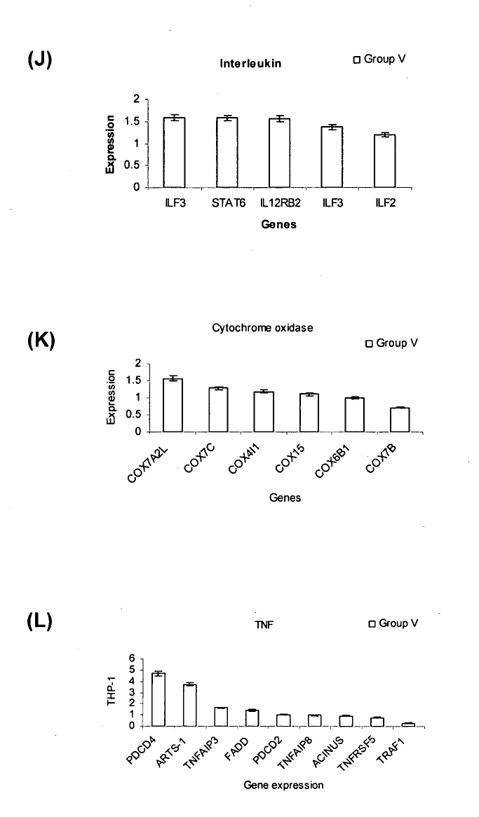

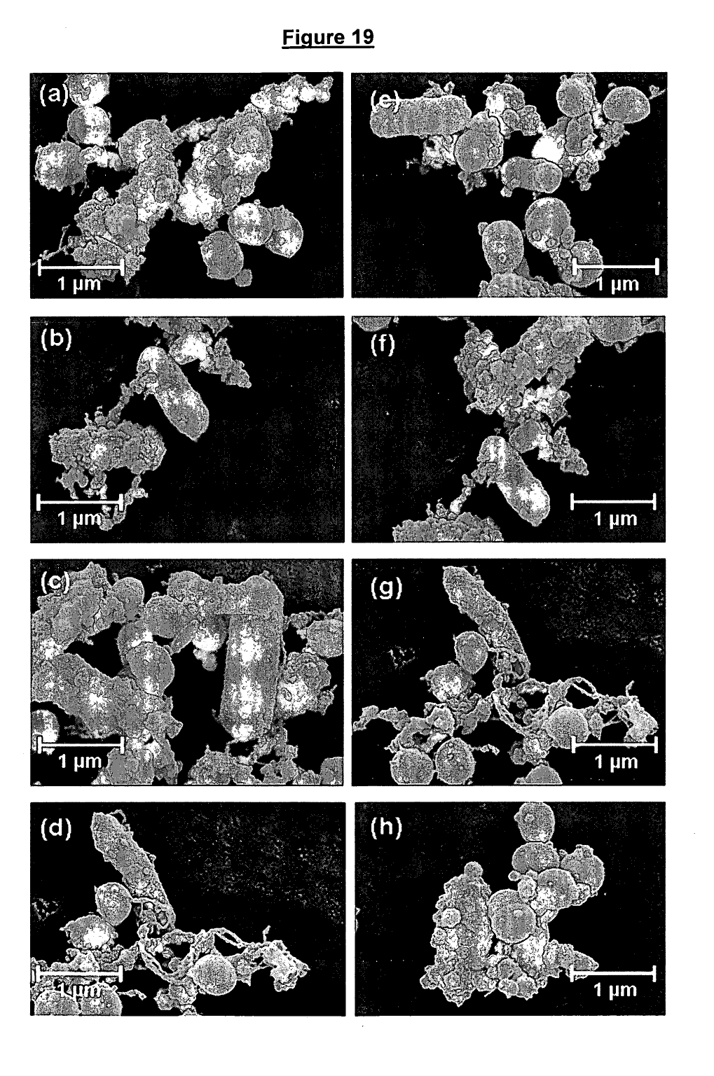

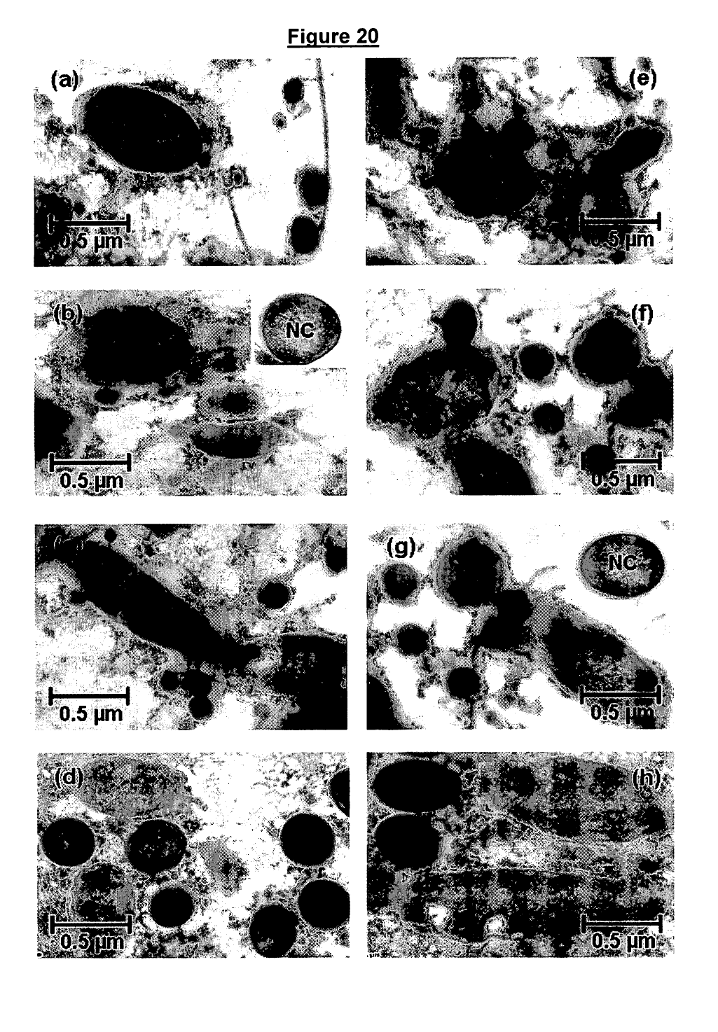

The SEM analysis (FIG. 19) revealed that the untreated bacterial (Staphylococcus aureus) cells had normal smooth surface morphology. In particular, Staphylococcus aureus bacteria treated with DRR2-PLA₂ showed pronounced changes in their morphology. DRR2-PLA₂ treated S. aureus bacteria showed mostly debris on the membrane, significant wrinkling surface, roughening, and membrane blebbing respectively. FIG. 20 showed the antimicrobial effect of DRR2-PLA₂ against Staphylococcus aureus at 5.12 μM concentration when compared to negative controls. It can be seen from the figures that S. aureus treated with DRR2-PLA₂ showed pore formation, cell wall thickening, mostly debris on the membrane, significant wrinkling surface, roughening, and membrane blebbing respectively.

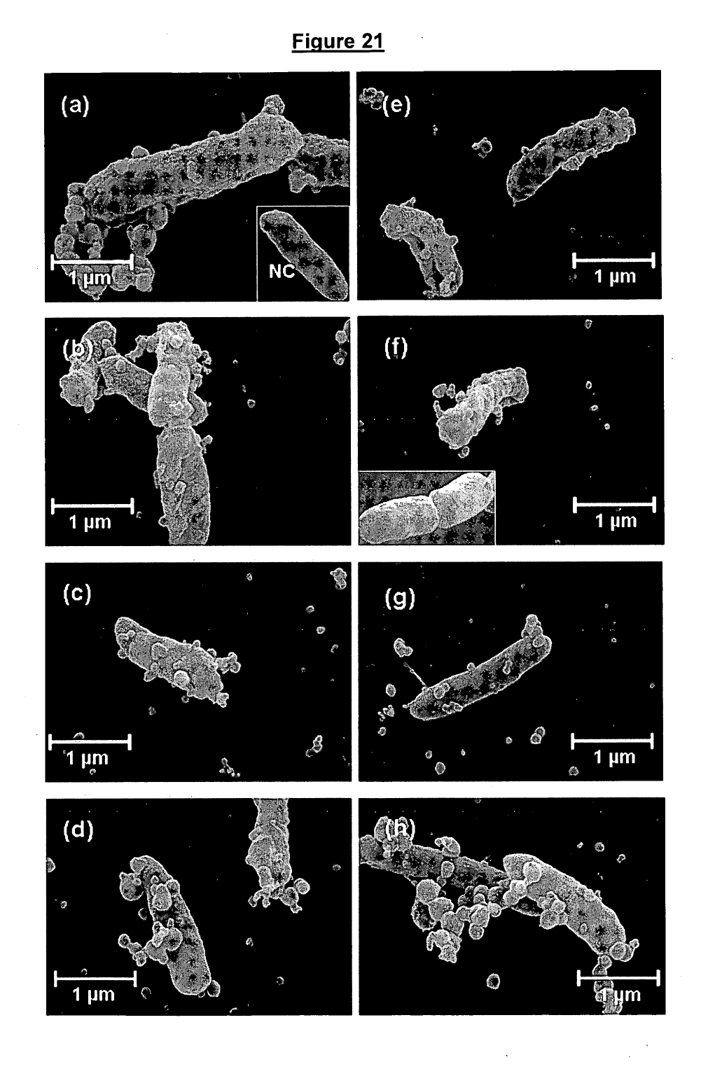

The SEM picture of FIG. 21 revealed that the untreated bacterial (Proteus vulgaris) cells had normal smooth surface morphology. Proteus vulgaris bacteria treated with DRR2-PLA₂ showed pronounced changes in their morphology. DRR2-PLA₂ treated P. vulgaris bacteria showed mostly debris on the membrane, significant wrinkling surface, roughening, and membrane blebbing respectively.

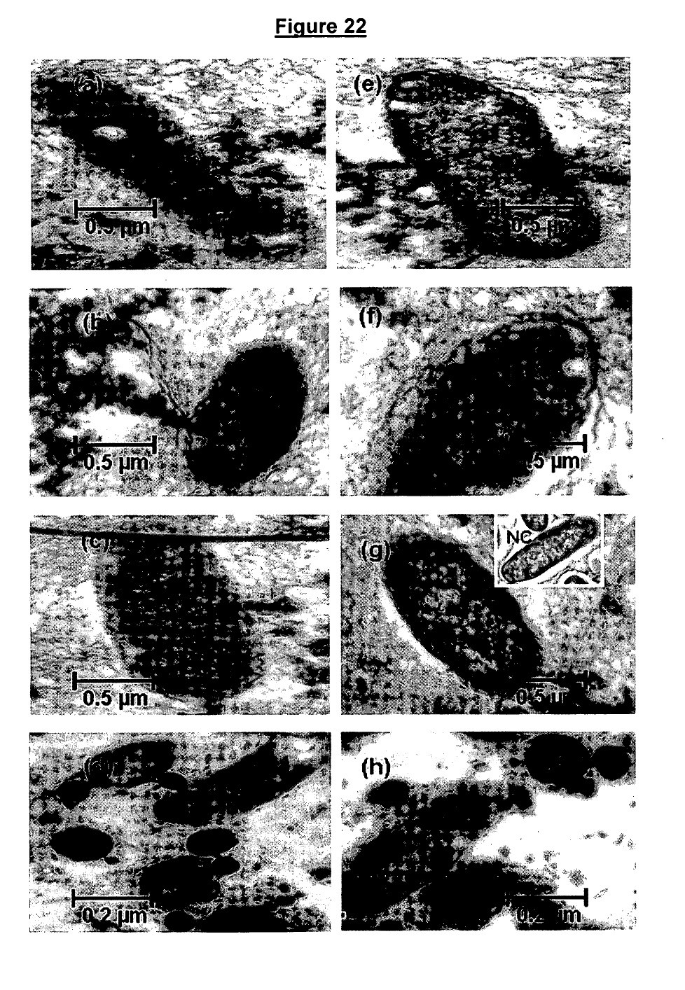

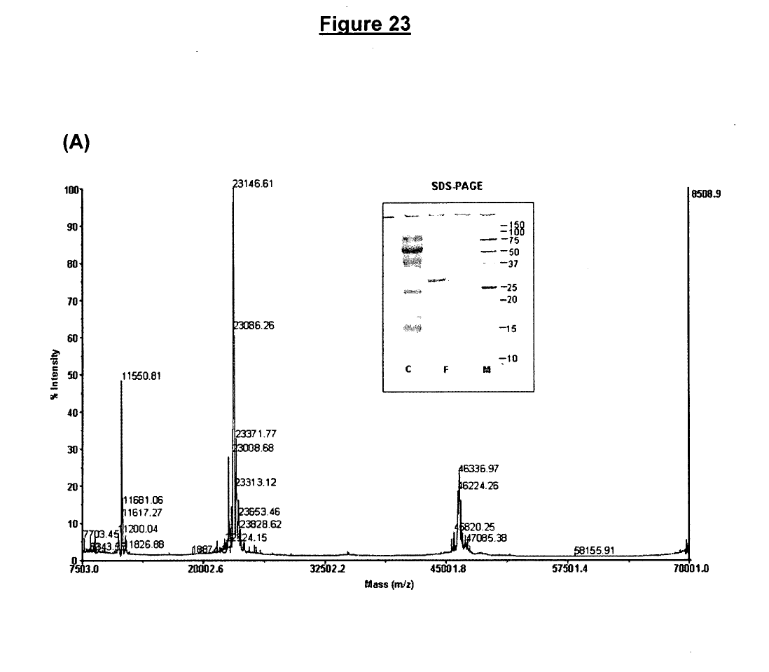

The clear architecture of the S. aureus cell wall and internal details in the control bacteria are shown in FIG. 22. FIGS. 22(b) and (c) revealed striking structural alterations in S. aureus exposed to DRR2-PLA₂. Detailed morphology of normal P. vulgaris bacteria as a control is shown in FIG. 22(d). It can be seen from FIG. 22(e) that treatment with AH-PLA₂ resulted in numerous mushroom-shaped blebs, retraction of cytoplasm and apparent loss of cell contents particularly at the division septa (P. vulgaris), whereas FIG. 22(f) Proteus mirabilis treated with DRR2-PLA₂ resulted in irregular shape bacterial cell wall and membrane damage as shown in the microscopy.

### CONCLUSION

In the present study, DRR2-PLA₂ showed most potent bactericidal activity than the DRR1-PLA₂, the mechanism of action was proved by ultra-structural studies. The DRR2-PLA₂ has induced pore formation on Gram-negative bacteria. Whereas, the invasion of cytotoxicity assay, 0.1 mM concentrations kill the bacteria but the monocytic (THP-1, human macrophage) cells did not show any harmful effect at the same dose. The based on the above results, the new novel DRR2-PLA₂ is neither cytotoxic on monocytic cells (THP-1) nor hemolytic on human erythrocytes. The microarray analysis data showed that the greatest number of up-regulated genes (114 out of 2912) was secreted proteins, including cytokines (COX) and tumor necrosis factor alpha (TNF-alpha) known to be induced by bacterial stimulation of macrophages. The strongest up-regulation was recorded for interlukin-6 (STAT-6, IL-12), interferon gamma (IFNγ) and chemokines compared to controls respectively. The extensive amount of data accumulated from the GeneChip study and the detail analysis (PCR & Western blot) will show us the exact mechanisms of action of this DRR2-PLA₂ and have it acts.

### REFERENCES

1. Amsterdam, D. Susceptibility testing of antimicrobials in liquid media. In: Lorian V. editor. Antibiotics in laboratory medicine. 4. Baltimore, Md.: Williams and Wilkins; 1996. pp. 52-111.

2. Bauer A W, Kirby W M, Sherris J. C. (1966) Antibiotic susceptibility testing by a standardized single disk method. Amer. J Clin Pathol 45, 493-496.

3. Blaylock R S M. Antibacterial properties of KwaZulu Natal snake venoms. Toxicon. 2000; 38:1529-1534.

4. Bonventre J. V., Huang Z., Reza Taheri M., O'Leary E., Li E., Moskovitz M. A. et al. (1997) Reduced fertility and postischaemic brain injury in mice deficient in cytosolic phospholipase A2. Nature 390: 622-625.

5. Bouchier C, Boulain J C, Bon C, Menez A. Analysis of cDNAs encoding the two subunits of crotoxin, a phospholipase A₂ neurotoxin from rattlesnake venom: the acidic non enzymatic subunit derives from a phospholipase A₂-like precursor. Biochem Biophys Acta. 1991; 1088:401-408.

6. Bradford M M. A rapid and sensitive method for quantification of microgram quantities of protein utilizing the principle of protein dye binding. Ann Biochem. 1976; 72:248-254.

7. Chakrabarty, D., Bhattacharyya, D., Sarkar, H. and Lahiri, S. C. (1993) Purification and partial characterization of a haemorrhagin (VRH-I) from Vipera russelli russelli venom. Toxicon 31, 1601 1614.

8. Dance D A B, Melioidosis. In Manson's tropical diseases 20^{th} edition. Edited by: Cook G C. London, England: W. B. Saunders Co. Ltd; 1996:925-930.

9. Eickhoff T C et al, Pseudomonas pseudomallei: susceptibility to chemotherapeutic agents, J Infect Dis, 1970, 121:95-102.

10. Elsinghorst, E. A., (1994). Measurement of invasion by gentamicin resistance. Methods in Enzymology 236, 405-420.

11. Gowda, T. V. and Middlebrook, J. L. (1993) Effects of myonecrotic snake venom phospholipase A 2 toxins on cultured muscle cells. Toxicon 31, 1267-1278.

12. Gowda, T. V. and Middlebrook, J. L. (1994) Monoclonal antibodies to VRV-PL-VIIIa, a basic multitoxicphospholipase A 2 from Vipera russelli venom. Toxicon 32, 955464.

13. Gowda, T. V., Schmidt, J. and Middlebrook, J. L. (1994) Primary sequence determination of the most basic myonecrotic phospholipase A2 from the venom of Vipera russelli. Toxicon 32, 665-673.

14. Hancock R E, Fall T, Brown M. (1995) Cationic bactericidal peptides. Advance Microb Physiol 37, 135-175.

15. Harris, J. B. and Faiz, M. A. and Vater, R. (1994) Muscle damage caused by the venoms of snakes with particular reference to Russell's viper. Toxicon 32, 523-524.

16. Heng B H et al, Epidemiological surveillance of melioidosis in Singapore, Ann Acad Med Singapore, 1998, 27(4):478-484.

17. Huang, H. C. and Lee, C. Y. (1984) Isolation and pharmacological properties of phospholipases A 2 from Viperarusselli (Russell's viper) snake venom. Toxicon 22, 207-217.

18. Jayanthi, G. P. and Gowda, T. V. (1989) Dissociation of catalytic activity and neurotoxicity of a basic phospholipase A 2 from Russell's viper (Vipera russelli) venom. Toxicon 27, 875-885.

19. Jeyaseelan K, Armugam A, Donghui M, Tan N H. Structure and phylogeny of the venom group I phospholipase A(2) gene. Mol Biol Evol. 2000 Jul; 17(7):1010-21.

20. Jones A L et al, Intracellular survival of Burkholderia pseudomallei, Infect Immu, 1996, 64:782-790.

21. Kasturi, S. and Gowda, T. V. (1989) Purification and characterization of a major phospholipase A 2 from Russell's viper (Vipera russelli) venom. Toxicon 27, 229-237.

22. Kini R. M. (1997) Phospholipase A2—a complex multifunctional protein puzzle. In: Venom Phospholipase A2 Enzymes: Structure, Function and Mechanism, pp. 1-28, Kini R. M. (ed.), Wiley, Chichester.

23. Kini, R. M., Evans, H. J., (1989). A common cytolytic region in myotoxins, hemolysins, cardiotoxins and antibacterial peptides. Int J Pept Protein Res. 34(4), 277-86.

24. Kisiel, W. (1979) Molecular properties of the factor V-activating enzyme from Russell's viper venom. J. biol. Chem. 254, 12,230-12,234.

25. Krizaj, 1., Turk, D., Ritonja, A. and Gubensek, F. (1989) Primary structure of ammodytoxin C further reveals the toxic site of ammodytoxin. Biochem. biophys. Acta 999, 198-202.

26. Laemmli, U. K. (1970) Cleavage of structural protein during the assembly of the head of bacteriophage T4. Nature 227, 680-685.

27. Langer M, et al, Novel peptide conjugates for tumor-specific chemotherapy, J. Med Chem, 2001, 26, 44(9):1341-8.

28. Lee, C. Y. (1944) Toxicological studies on the venom of Vipera russelliiformosensis. IV. On the cause of death in rabbits. Folia pharmac. Jpn. 40, 53.

29. Leelarasamee A: Burkholderia pseudomallei: the unbeatable foe? Southeast Asian J Trop Med Public Health, 1998, 29:410-415.

30. Matsuzaki K., (1998) Magainins as paradigm for the mode of action of pore forming polypetides. Biochim Biophys Acta 1376, 391-400.

31. Motizuki M, Itoh T, Satoh T, Yokota S, Yamada M, Shimamura S, Samejima T, Tsurugi K., (1999) Lipid-binding and antimicrobial properties of synthetic peptides of bovine apolipoprotein A-II. Biochem J 342:215-221.

32. National Committee for Clinical Laboratory Standards. Performance Standards for Antimicrobial Disk Susceptibility Tests: Approved Standard. M2-A6. National Committee for Clinical Standards, Wayne, Pa. 1997.

33. National Committee for Clinical Laboratory Standards. Methods for Dilution Antimicrobial Susceptibility Tests for Bacteria that Grow Aerobically. Approved Standard M7-A4. National Committee for Clinical Standards, Wayne, Pa. 1997.

34. Oliveira D G, Toyama M H, Novello J C, Beriam L O S, Marangoni S. Structural and functional characterization of basic PLA₂ isolated from Crotalus durissus terrificus venom. J Protein Chem. 2002; 21:161-168.

35. Ombregt et al., A System of Orthopaedic Medicines, Elsevier Health Sciences, 2003, page 39.

36. Oscar Cirioni et al, Antimicrobial Agent and Chemotherapy, 2002, 46(1):101-104.

37. Pachiappan, A., Thwin, M. M., Manikandan, J., Gopalakrishnakone, P., (1995) Glial inflammation and neurodegeneration induced by candoxin, a novel neurotoxin from Bungarus candidus venom: global gene expression analysis using microarray. Toxicon 46, 883-899.

38. Páramo L., Lomonte B., Pizarro-Cerdá J., Bengoechea J. A., Gorvel J. P., Moreno. E., (1998) Bactericidal activity of Lys-49 and Asp49 myotoxic phospholipases A₂ from Bothrops asper snake venom: synthetic Lys-49 myotoxin II-(115-129) peptide identifies its bactericidal region. European Journal of Biochemistry 253, 452-461.

39. Rajchanuvong A, Chaowagul W, Suputtamongkol Y, Smith M D, Dance D A B, White N J. A perspective comparison of co-amoxiclav and the combination of chloramphenicol, doxycycline and co-trimoxazole for the oral maintenance treatment of melioidosis. Br J Clin Pharmacology. 2000; 49:184-191.

40. Reynolds L J, Hughes L L, Dennis E A. Analysis of human synovial fluid phospholipase A₂ on short chain phosphatidycholine-mixed micelles: Development of a spectrophotometric assay suitable for a microtiterplate reader. Ann Biochem. 1992; 204:190-197.

41. Sahly H, Schubert S, Harder J, Rautenberg P, Ullmann U, Schröder J, Podschun R. Burkholderia is highly resistant to human Beta-defensin 3. Antimicrob Agents Chemother. 2003; 47:1739-1741.

42. Saiman L, Tabibi S, Starner T D, San Gabriel P, Winokur P L, Jia H P, McCray P B Jr, Tack B F. Cathelicidin peptides inhibit multiply antibiotic-resistant pathogens from patients with cystic fibrosis. Antimicrob Agents Chemother. 2001; 45:2838-2844.

43. Salach, J. I., Turini, P., Seng, R., Hauber, J. and Singer, T. P. (1971) Phospholipase A of snake venom. I.Isolation and molecular properties of isoenzymes from Naja naja and Vipera russelli venoms. J. biol. Chem. 246, 331-339.

44. Stiles B G, Sexton F W, Weinstein S A. Antibacterial effects of different snake venoms: purification and characterisation of antibacterial proteins from Pseudechis australis (Australian king brown or Mulga snake) venom. Toxicon. 1991; 29:1129-1141.

45. Takahashi, H., Iwanaga, S., Kitagawa, T., Hokama, Y. and Suzuki, T. (1974) Snake venom proteinase inhibitor II. Chemical structure of inhibitor-II isolated from the venom of Russell's viper (V. russelli). J. Biochem. 76, 721 733.

46. Takeya, H., Nishida, S., Miyata, T., Kawada, S. I., Saisaka, Y., Morita, T. and Iwanaga, S. (1992) Coagulation factor X activating from Russell's viper venom (RVV-X). J. biol. Chem. 267, 14,109-14,117.

47. Talan D A, Citron D M, Overturf G D, Singer B, Froman P, Goldstein E J. Antibacterial activity of crotalid venoms against oral snake flora and other clinical bacteria. J Infect Dis. 1991; 164:195-198.

48. Thwin M M, Gopalakrishnakone P, Yuen R, Tan C H. A major lethal factor of the venom of Burmese Reussell's viper (Daboia russelli siamensis): Isolation, N-terminal sequencing and biological activities of daboiatoxin. Toxicon. 1995; 33:63-76.

49. Travis, S. M., Anderson, N. N., Forsyth, W. R., Espiritu, C., Conway, B. D., Greenberg, E. P., McCray Jr. P. B., Lehrer, R. I., Welsh, M. J., Tack, B. F. (2000). Bactericidal activity of mammalian cathelicidin-derived peptides. Infection and Immunity 68(5), 2748-2755.

50. Tsai, I. H., Lu, P. J. and Su, Y. C. (1993) Russtoxin: a new family of two-component phospholipase A 2 toxins from Russell's vipers. In: Peptides: Chemistry and Biology, Proceedings of the 13th American Peptide Symposium, pp. 464-466 (Hodges, R. S. and Smith, J. A., Eds). The Netherlands: Escom Science.

51. Valentin, E., Lambeau, G., (2000). What can venom phospholipases A(2) tell us about the functional diversity of mammalian secreted phospholipases A(2)?. Biochimie. 82(9-10): 815-31.

52. van Deenen, L. L. M., de Haas, G. H., (1963). The substrate specificity of phospholipase A₂. Biochem. Biophys. Acta 70, 538-553.

53. Vishwanath, B. S., Kini, R. M. and Gowda, T. V. (1988) Purification and partial biochemical characterization of an edema inducing phospholipase A 2 from Vipera russelli (Russell's viper) snake venom. Toxicon 26, 721-731.

54. Wang, Y. M., Lu, P. J., Ho, C. L. and Tsai, I. H. (1992) Characterization and molecular cloning of neurotoxic phospholipase A 2 from Taiwan viper (Vipera russelli formosensis). Eur. J. Biochem. 209, 635-641.

55. White N J, Dance D A, Chaowagul W, Wattanagoon Y, Wuthiekanun V, Pitakwatchara N. Halving of mortality of severe melioidosis by ceftazidime. Lancet. 1989; 2:697-701.

56. Woodhams, B. J., Wilson, S. E., Bao Cheng Xin and Hutton, R. A. (1990) Differences between the venoms of two sub-species of Russell's viper: Vipera russelli pulchella and Vipera russelli siamensis. Toxicon 28, 427-433.

What is claimed is:

1. A method for the treatment and/or prevention of a bacterial related condition comprising administering to a subject a therapeutically effective amount of at least one phospholipase, isoform, derivative, mutant and/or fragment thereof.

2. The method of claim 1, wherein the bacterial related condition comprises at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli, with the proviso that the condition is not caused by Staphylococcus aureus and/or Escherichia coli when the phospholipase, isoform, derivative, mutant and/or fragment thereof is myotoxin II or BnpTx I.

3. The method according to claim 1, wherein the at least one phospholipase is a secretory or cytoplasmic phospholipase.

4. The method according to claim 3, wherein the secretory phospholipase is pancreatic, synovial and/or venomous phospholipase.

5. The method according to claim 1, wherein the phospholipase, isoform, derivative, mutant and/or fragment thereof, is from venom of Daboia russelli russelli, Daboia russelli siamensis, Daboia russelli pulchella, Crotalus adamanteus, Crotalus durissus terrificus, Pseudechis australis, Agkistrodon halys, Pseudechis guttata, Bitis arietans, Bitis gabonica rhinoceros, Echis carinatus, Acanthopis antarticus, Bungarus candidus, Bothrops asper, Bothrops jararacussu and/or Apis mellifera.

6. The method according to claim 1, wherein the phospholipase is phospholipase A₂.

7. The method according to claim 1, wherein the phospholipase, isoform, derivative, mutant and/or fragment thereof comprises at least one amino acid selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13 and SEQ ID NO:14.

8. The method according to claim 1, wherein the phospholipase, isoform, derivative, mutant and/or fragment thereof comprises at least one amino acid substitution, addition, deletion and/or at least one chemical modification.

9. The method according to claim 1, wherein the condition is melioidosis.

10. The method according to claim 1, wherein the treatment and/or prevention comprises administering the phospholipase, isoform, derivative, mutant and/or fragment thereof:
at least once and/or continuously, before the onset of the condition;
at least once and/or continuously, during the onset of the condition; and/or
at least once and/or continuously, after the onset of the condition.

11. The method according to claim 1, wherein the phospholipase, isoform, derivative, mutant and/or fragment thereof, is administered in conjunction with at least one pharmaceutically acceptable excipient, diluent, carrier and/or adjuvant.

12. The method according to claim 1, wherein the subject is a mammal.

13. The method according to claim 12, wherein the mammal is human.

14. A pharmaceutical composition formulated for the treatment and/or prevention of a bacterial related condition, wherein the composition comprises: a therapeutically effective amount of: a phospholipase, isoform, derivative, mutant and/or fragment thereof; and/or at least one pharmaceutically acceptable excipient, diluent, carrier and/or adjuvant.

15. The composition according to claim 14, wherein the bacterial related condition comprises at least one condition induced by at least one of the following: Burkholderia pseudomallei, Proteus vulgaris, Enterobacter aerogenes, Proteus mirabilis, Pseudomonas aeruginosa, Staphylococcus aureus and Escherichia coli, with the proviso that the condition is not caused by Staphylococcus aureus and/or Escherichia coli when the phospholipase, isoform, derivative, mutant and/or fragment thereof is myotoxin II or BnpTx I.

16. The composition according to claim 14, wherein the phospholipase, isoform, derivative, mutant and/or fragment thereof, is from at least one venom selected from the group consisting of: Daboia russelli russelli, Daboia russelli siamensis, Daboia russelli pulchella, Crotalus adamanteus, Crotalus durissus terrificus, Pseudechis australis, Agkistrodon halys, Pseudechis guttata, Bitis arietans, Bitis gabonica rhinoceros, Echis carinatus, Acanthopis antarticus, Bungarus candidus, Bothrops asper, Bothrops jararacussu and Apis mellifera.

17. The composition according to claim 14, wherein the phospholipase is phospholipase A₂.

18. The composition according to claim 14, wherein the phospholipase, isoform, derivative, mutant and/or fragment thereof comprises at least one amino acid selected from the group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13 and/or SEQ ID NO:14.

19. A kit for the treatment and/or prevention of a bacterial related condition comprising a phospholipase, isoform, derivative, mutant and/or fragment thereof, and optionally at least one pharmaceutically acceptable excipient, diluent, carrier and/or adjuvant.

20. A method for the treatment and/or prevention of a bacterial related condition comprising administering to a subject a therapeutically effective amount of at least one phospholipase, isoform, derivative, mutant and/or fragment thereof comprising the amino acid sequence of SEQ ID NO:1 and/or SEQ ID NO:2.

21. The method according to claim 20, wherein the bacterial related condition comprises at least one condition induced by at least one of the following Burkholderia pseudomallei, Proteus vulgaris, Enterobacter aerogenes, Proteus mirabilis, Pseudomonas aeruginosa, Staphylococcus aureus and Escherichia coli.

22. An isolated peptide comprising at least one amino acid sequence selected from the group consisting of: SEQ ID NO:1, SEQ ID NO:2, an isoform, derivative, mutant and/or fragment thereof.

23. The peptide of claim 22, wherein the peptide is a fused peptide and comprises at least one peptide comprising the sequence of SEQ ID NO:1 and/or SEQ ID NO:2.

24. The peptide of claim 22, wherein the peptide is isolated and/or purified from venom.

25. The peptide of claim 22, wherein the venom is from Daboia russelli russelli.

26. The peptide of claim 22, wherein the molecular weight of the peptide is 13822 Da or 13669 Da.


SEQUENCE LISTING (14 sequences)

SEQ ID NO: 1 - protein, 28 aa, N-terminal sequence of DRR1-PLA2 from Daboia russellii russelli
SLLGFGCMILEETGVMIELEKNCNQHPE

SEQ ID NO: 2 - protein, 37 aa, N-terminal sequence of DRR2-PLA2 from Daboia russellii russelli
SLLEFGMMILEETGKLAVPFYSKYGLYCGCGGKTPDD

SEQ ID NO: 3 - protein, 122 aa, N-terminal sequence of crotoxin basic chain 1 from Crotalus durissus terrificus
HLLQFNKHIKFETRKNAIPFYAFYGCYCGWGGRGRPKDATRDCCFVHDCCYGKLAKCNTKWDIYPYSLKSGYITCGKGTWCEEQICECDRVAAECLRRSLSTYKYGYHFYPDSRCRGPSETC

SEQ ID NO: 4 - protein, 122 aa, N-terminal sequence of crotoxin basic chain 2 from Crotalus durissus terrificus
SLLQFNKMIKFETRKNAVPFYAFYGCYCGWGGQGRPKDATDRCCFVHDCCYGKLAKCNTKWDIYRYSLKSGYITCGKGTWCKEQICECDRVAAECLRRSLSTYKNEYMFYPDSRCREPSETC

SEQ ID NO: 5 - protein, 122 aa, N-terminal sequence of Agkistrotoxin from Agkistrodon halys
NLLQFNKMIKEETGKNAIPFYAFYGCYCGGGGQGKPKDGTDRCCFVHDCCYGRLVNCNTKSDIYSYSLKEGYITCGKGTNCEEQICECDRVAAECFRRNLDTYNNGYMFIRDSKCTETSEEC

SEQ ID NO: 6 - protein, 116 aa, N-terminal sequence of VRV-PL-VIIIa from Daboia russellii pulchella
LLEFGKMILEETGKLAIPSYSSYGCYCGWGGKGTPKDATDRCCFVHDCCYGNLPDCNPKSDRYKYKRVNGAIVCEKGTSCENRICECDKAAAICFRQNLNTYSKKYMLYPDFLCKG

SEQ ID NO: 7 - protein, 121 aa, N-terminal sequence of myotoxin I from Bothrops asper (Terciopelo)
LIEFAKMILEETKRLPFPYYTTYGCYCGWGGQGQPKDATDRCCFVHDCCYGKLSNCKPKTDRYSYSRKSGVIICGEGTPCEKQICECDKAAAVCFRENLRTYKKRYMAYPDLLCKKPAEKC

SEQ ID NO: 8 - protein, 121 aa, N-terminal sequence of RVV-VD from Daboia russellii russelli
NLFQFAEMIVKMTGKNPLSSYSDYGCYCGWGGKGKPQDATDRCCFVHDCCYEKVKSCKPKLSLYSYSFQNGGIVCGDNHSCKRAVCECDRVAATCFRDNLNTYDKKYHNYPPSQCTGTEQC

SEQ ID NO: 9 - protein, 122 aa, N-terminal sequence of RV-4 precursor from Daboia russellii siamensis
NLFQFARMINGKLGAFSVWNYISYGCYCGWGGQGTPKDATDRCCFVHTCCYGGVKGCNPKLAYICYSFQRGNIVCGRNNGCLRTICECDRVAANCFHQNKNTYNKEYKFLSSSKCRQRSEQC

SEQ ID NO: 10 - protein, 115 aa, N-terminal sequence of myotoxin II from Bothrops asper
LFELGKMILQETGKNPAKSYGAYGCNCGVLGRGKPKDATDRCCYVHKCCYKKLTGCNPKKDRYSYSWKDKTIVCGENNSCLKELCECDKAVAICLRENLNTYNKKYRYYLKPLCK

SEQ ID NO: 11 - protein, 115 aa, N-terminal sequence of BthTX-I from Bothrops jararacussu
LFELGKMILQETGKNPAKSHGAYGCNCGVLGRGKPKDATDRCCYVHKCCYKKLTGCDPKKDRYSYSWKDKTIVCGENNPCLKELCECDKAVAICLRENLGTYNKKYRYHLKPFCK

SEQ ID NO: 12 - protein, 121 aa, N-terminal sequence of Ecarpholin S from Echis carinatus
VVELGKMIIQETGKSPFPSYTSYGCFCGGGERGPPLDATDRCCLAHSCCYDTLPDCSPKTDRYKYKRENGEIICENSTSCKKRICECDKAVAVCLRKNLNTYNKKYTYYPNFWCKGDIEKC

SEQ ID NO: 13 - protein, 27 aa, N-terminal sequence of PLA2 enzyme from Agkistrodon halys
HLLQFRKMIKKMTGKEPVVSYAFYGCY

SEQ ID NO: 14 - protein, 20 aa, N-terminal sequence of PLA2 enzyme from Agkistrodon halys
IVSPPVCGNELLEVGEECDD